SEQUENCE LISTING (54 sequences)

SEQ ID NO: 1 - DNA, 9215 bp, Homo sapiens
gcggccgccccgagacgccggccccgctgagtgatgagaacagacgtcaaactgccttatgaatattgatgcggaggctaggctgctttcgtagagaagcagaaggaagcaagatggctgccctttaggatttgttagaaaggagacccgactgcaactgctggattgctgcaaggctgagggacgagaacgaggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtaggtaagtgccagaaatgtactgagacacatggcgtttatccagaattagcaaatttatcttcagatatgggattttccttctttttttaaatcttgagtctggcagcaatttgtaaaggctcataaaaatctgaagcttacattttttgtcaagttaccgatgcttgtgtcttgtgaaagagaacttcacttacatgcagtttttccaaaagaattaaataatcgtgcatgtttatttttccctctcttcagatcctgtaaaatttgaatgtatctgttttagatcaattcgcctatttagctctttgtatattatctcctggagagacagctaggcagcaaaaaaacaatctattaaaatgagaaaataacgaccataggcagtctaatgtacgaactttaaatattttttaattcaaggtaaaatatattagtttcacaagatttctggctaatagggaaattattatcttcagtcttcatgagttgggggaaatgataatgctgacactcttagtgctcctaaagtttccttttctccatttatacatttggaatgttgtgatttatattcattttgattcccttttctctaaaatttcatctttttgattaaaaaatatgatacaggcatacctcagagatattgtgggtttggctccataccacaataaaatgaatattacaataaagcaagttgtaaggactttttggtttctcactgtatgtaaaagttatttatatactatactgtaacatactaagtgtgcaatagcattgtgtctaaaaaatatatactttaaaaataatttattgttaaaaaaatgccaacaattatctgggcctttagtgagtgctaatctttttgctggtggagggtcgtgcttcagtattgatcgctgtggactgatcatggtggtagttgctgaaggttgctgggatggctgtgtgtgtggcaatttcttaaaataagacaacagtgaagtgctgtatcaattgatttttccattcacaaaagatttctctgtagcatgcaatgctgtttgatagcatttaacccacagcagaatttctttgaaaattggactcagtcctctcaaactgtgctgctgctttatcaactaagtttttgtaattttctgaatcctttgttgtcatttcagcagtttacagcatcttcattggaagtatattccatctcaaacattctttgttcatccataagaagcaacttcttatcaagttttttcatgacattgcagtaactcagccccatcttcaggctctacttctaattctggttctcttgctacatctccctcatctgcagtgacctctccacggaagtcttgaactcctcaaagtaatccatgagggttggaatcaacttctaaactcctgttaatgttgatatattgaccccctcccatgaattatgaatgttcttaataacttctaaatggtgatacctttccagaaggctttcaatgtactttgcccggatccatcagaagactatcttggcagctgtagactaacaatatatttcttaaatgataagacttgaaagtcaaaagtactccttaatccataggctgcagaatcaatgttgtattaacaggcacgaaaacagcattaatcttgtgcatctccatcggagctcttgggtgactaggtgccttgagcagtaatattttgaaaggaggttttggttttgttttttgttttttttttttgttttttagcagtaagtctcaacactgggcttaaaatattcagtaaactatgttgtaaaaagatgtgttatcatccagactttgttgttccattactctacacaagcagggtacacttagcataattcttaagggccttggaattttcagaatggtaaatgagtatgggcttcaacttaaaatcatcaactgcattagcctgtaacaagagagtcagcctgtcctttgaagcaaggcattgacttctatctatgaaagtcttagatggcaccttgtttcaatagtaggctgtttagtacagccaccttcatcagtgatcttagctagatcttctgcataacttgctgcagcttctacatcagcacttgctgcctcaccttgtccttttatgttatagagacagctgcgcttcttaaactttataaaccaacttctgctagcttccaacttctcttctgcagcttcctcattctcttcatagaactgaagggagtcaaggccttgctctggattaagctttggcttaaggaatgttgtggctgacgtgatcttctatccagaccactaaagcgctctccatatcagcaataaggccgttttgctttcttacctttcatgtgttcactggagtaatttccttcaagaatttttcctttacattcacaacttggctaactggcatgcaaggcctagctttcagcctgtcttggcttttgacatgccttcctcacttagctcgtcatatctagcttttgatttaaagtggcaggcatacaactcttcctttcacttgaacacttagaggccactgtagggttattaattggcctaatttcaatattgttgtgttttagggaatagagaggcccagggagagggagagagcccaaacggctggttgatagagcaggcagaatgcacacaacatttatcagattatgtttgcaccatttaccagattatgggtacggtttgtggcaccccccaaaaattagaatagtaacatcaaagatcactgatcacagatcgccataacataaataataataaactttaaaatactgtgagaattaccaaaatgtgatacagagacatgaagtgagcacatgctgttgaaaaaaatgacactgatagacatacttaacacgtgggattgccacaaaccttcagtttgtaaaagtcacagtaactgtgactcacaaaagaacaaagcacaataaaacgaggtatgcctgtatttttaaaaaaagctttttgttaaaattcaggatatgtaataggtctgtaggaatagtgaaatatttttgctgatggatgtagatatatacgtggatagagatgaagatcttaattatagctatgcagcatagatttagtcaaagacatttgaaaagacaaatgttaaattagtgtggctaatgacctacccgtgccatgttttccctcttgcaatgagataccccacactgtgtagaaggatggagggaggactcctactgtccctctttgcgtgtggttattaagttgcctcactgggctaaaacaccacacatctcatagataatatttggtaagttgtaatcgtcttcactcttctcttatcacccacccctatcttcccacttttccatctttgttggtttgcaacagccccttctttttgcctgactctccaggattttctctcatcataaattgttctaaagtacatactaatatgggtctggattgactattcttatttgcaaaacagcaattaaatgttatagggaagtaggaagaaaaaggggtatccttgacaataaaccaagcaatattctgggggtgggatagagcaggaaattttatttttaatcttttaaaatccaagtaataggtaggcttccagttagctttaaatgttttttttttccagctcaaaaaattggattgtagttgatactacatataatacattctaattccctcactgtattctttgtttagtttcatttatttggtttaaaataattttttatcccatatctgaaatgtaatatatttttatccaacaaccagcatgtacatatacttaattatgtggcacattttctaatagatcagtccatcaatctactcattttaaagaaaaaaaaattttaaagtcacttttagagcccttaatgtgtagttgggggttaagctttgtggatgtagcctttatatttagtataattgaggtctaaaataataatcttctattatctcaacagagcaaattattgaaaaagatgaaggtcctttttatacccatctaggagcaggtcctaatgtggcagctattagagaaatcatggaagaaaggtaattaacgcaaaggcacagggcagattaacgtttatccttttgtatatgtcagaatttttccagccttcacacacaaagcagtaaacaattgtaaattgagtaattattagtaggcttagctattctagggttgccaacactacacactgtgctattcaccagagagtcacaatatttgacaggactaatagtctgctagctggcacaggctgcccactttgcgatggatgccagaaaacccaggcatgaacaggaatcggccagccaggctgccagccacaaggtactggcacaggctccaacgagaggtcccactctggctttcccacctgataataaagtgtcaaagcagaaagactggtaaagtgtggtataagaaaagaaccactgaattaaattcacctagtgttgcaaatgagtacttatctctaagttttcttttaccataaaaagagagcaagtgtgatatgttgaatagaaagagaaacatactatttacagctgcctttttttttttttttcgctatcaatcacaggtatacaagtacttgcctttactcctgcatgtagaagactcttatgagcgagataatgcagagaaggcctttcatataaatttatacagctctgagctgttcttcttctagggtgccttttcattaagaggtaggcagtattattattaaagtacttaggatacattggggcagctaggacatattcagtatcattcttgctccatttccaaattattcatttctaaattagcatgtagaagttcactaaataatcatctagtggcctggcagaaatagtgaatttccctaagtgccttttttttgttgtttttttgttttgttttttaaacaagcagtaggtggtgctttggtcataagggaagatatagtctatttctaggactattccatattttccatgtggctggatactaactatttgccagcctccttttctaaattgtgagacattcttggaggaacagttctaactaaaatctattatgactccccaagttttaaaatagctaaatttagtaagggaaaaaatagtttatgttttagaagactgaacttagcaaactaacctgaattttgtgctttgtgaaattttatatcgaaatgagctttcccattttcacccacatgtaatttacaaaatagttcattacaattatctgtacattttgatattgaggaaaaacaaggcttaaaaaccattatccagtttgcttggcgtagacctgtttaaaaaataataaaccgttcatttctcaggatgtggtcatagaataaagttatgctcaaatgttcaaatattt

SEQ ID NO: 2 - DNA, 4349 bp, Homo sapiens
gcccccgcctcgcgccgcgcgcacgtgactgcgcccccggccccgcccccggcctgccccccgcccccactggccgctcggccgcgcgcgggtcggccggctctatggggaaagcggcggctccgagccgaggcggcggctgtggcggccgctcccgcgggctctcgtcgctgttcacggttgtcccctgcctgtcgtgccacacggcggcgccgggcatgagcgcttccacgtccggctccgggccggagcccaagccccagccccagcccgtgcccgaaccggagcggggaccgctgtcagaacaggtgtcggaggcagtttcggaggcagtgccaagatcggaacctgtgtccgagacgacgtctgagccggagccaggggctgggcagccatcggaactgctgcaggggtcgcggccggggtcagagtcaagctcaggtgtaggggctgggcccttcactaaggccgcatcggagccgctctcccgggcggtggggagcgcgacctttctcagacccgagtcaggatcgctgccagcgttaaagcccctgcctcttctgcgaccaggacaggcgaagactcctcttggggttccaatgtcggggactggcacgacctccagtgccccactggccttactgcctctggacagcttcgagggctggcttctcaagtggaccaactatctgaagggctaccagcgccgctggttcgtgctgggcaatggtttgctctcttactacagaaatcagggtgaaatggcccacacgtgccgtggaaccatcaacctgtccaccgcgcacattgacacggaggactcttgtggtatcttgctgaccagtggggccaggagctaccacctcaaggccagctcagaggtggaccggcagcagtggatcaccgccctggagctggccaaggccaaggctgtccgcgtgatgaacactcattcagatgactctggggacgacgacgaggctaccaccccagccgacaagagcgagctgcaccacaccctgaagaatctttccctgaagttagatgacctcagcacgtgcaatgacctcatcgccaagcacggcgctgcactccagcgctccctgacagagctggacggcctcaagatcccatctgagagtggggagaagctgaaggtggtgaatgagcgggccaccctcttccgcatcacatccaatgctatgatcaacgcctgcagggacttcttggaactagcagagatacacagtcggaaatggcagcgggcactgcagtatgagcaggagcagcgcgtgcacttggaggaaaccattgagcagctggcgaagcagcacaacagcctcgagcgggccttccacagtgcccctggccggccggccaacccctccaagagcttcattgagggaagcctcttgactcccaaaggagaggacagtgaggaagatgaagataccgagtactttgatgccatggaagactccacatccttcatcaccgtgatcaccgaggccaaggaagacagcagaaaagctgaaggtagcaccgggacaagttccgtggactggagctcagcagacaatgtactagatggtgcctcgctcgtgcccaagggttcatccaaagtcaagaggcgagtccgcattcccaacaagcccaactacagccttaacctctggagcatcatgaagaactgcatcggccgggagctctccaggatccccatgccggtgaacttcaatgagcccctgtccatgctccagcggctgacagaggacctggagtaccaccacctgctggacaaggcagtgcactgcaccagctcagtggagcagatgtgcctggtggccgccttctctgtgtcctcctactccaccacagtgcaccgcatcgccaagcccttcaaccccatgctgggggagaccttcgagctggaccgcctcgacgacatgggcctgcgctccctctgtgagcaggtgagccaccaccccccctcagctgcgcactacgtgttctccaagcatggctggagcctctggcaggagatcaccatctccagcaagttccggggaaaatacatctccatcatgccgctaggtgccatccacttagaattccaggccagtgggaatcactacgtgtggaggaagagcacctcaactgttcacaacatcatcgtgggcaagctctggatcgaccagtcaggggacatcgagattgtgaaccataagaccaatgaccggtgccagctgaagttcctgccctacagctacttctccaaagaggcagcccggaaggtgacaggagtggtgagtgacagccagggcaaggcccattacgtgctgtccggctcgtgggatgaacaaatggagtgctccaaggtcatgcatagcagtcccagcagccccagctctgacgggaagcagaagacagtgtaccagaccctgtcagccaagctgctgtggaagaagtacccgctgccggagaacgcggagaacatgtactacttctcagagctggccctgaccctcaacgagcacgaggagggcgtagcgccaaccgacagccgcctgcggcccgaccagcggctgatggagaagggccgttgggacgaggccaataccgagaagcagcggctggaggagaagcagcgcctgtcgcggcgccggcggctggaggcctgcgggccgggcagcagctgcagctcggaggaagagaaggaggcggatgcctacacgccactgtggtttgagaagaggctggatccgctgaccggggagatggcctgtgtgtacaagggcggctactgggaggccaaggagaagcaagactggcatatgtgccccaacatcttctgagcgccacccttgcaacaaatacaggcgcctgcacagcctggcccacctgttcattaatgcactcaatttagtactgaatggtctttctcccagcccattcccagcccttcctatttcctttcctattttttttttctccccacactttcttgggactcccaccttggaaggaggaagggctgacctgggttctctccagcccccaggtgcgccgggtcacccgtgccccttcattatggacctgggccctaccggaacccctgccccagttaccacaactcaggccggctggcccgggccatgggctgcgcaaatcaccagcccccaacccagggaggaactggcccctcctagggagcctcttcgacttttttagaaaaatgatctccatttctttccagccatgatgtttagtaaatatttttagtaccgcacttagcagacagctttccaagtgtgctttcttgccacaaaagtgtcctggcaagagccccttatttttaagacatcaggaagccagaccgctttgagttgggagaattttgtagctcaacatatcaagtcctcgatggtatctgagctgcccacacccccacctgccaaggccccacagagcccaaaacagaagggggctgccccagcccagcagagcacagagtttctggagctcccatccacagatgcaggagggggtactgatggtaacccccatgtggatttgagggcagcagtccctggcctcaccctagccagcctgggtggctccctagccccaagaggccaggaagggctggaaggcagggcctgcaggtgctccccgccctgagacccaggccccaaatcagcaataatgaacaaacccttggcccagcctgggctggtgacctgggcaccagagaccttgcatccctcctcatcctaggaggcccctaggggtgccccatctcagtgtcccctgaactctttatttgcctaatttatatatatatatatgagatatataaatatatataaaatagctattttgcttaaatttctacagtatgtaaaagtgaaaaaatgatgaagacgggtgcacctgtctgagtttggccctcatgtgagctgtgcccttccctctcctcatgcccccttccagcggcttctgccaaccatggggggctggaccaccatggccactgacccagcccctcagaatcccacactccaatcctttccatttcagtttagtcctaaaagttcatcacagggtctttctttctactccaggactggttttgtttttatatatataaaaaaaaaaagtgaaaacaccaatgtgtgaaatgccttacaatgcccactggagaggcggggcggggtggggcaggatggccccactggggctcctacagagctgtggaatgtacctctccccaacactgttttgttagcgagcaccttttgaccagtaataaaaaaccttggctttggagttttccactgaaaaaaaaa

SEQ ID NO: 3 - DNA, 6076 bp, Homo sapiens
gattcctgcagggagggaggagagaagggcggcagtgggagggggaggtacctggaactgggagtgatgtcagctcccagctcggtgcctgcccggattcctgacatggtgtagtgcaggcagggtggggaaaggacggggaaggactcgtgtgctgcgagctggcggccgggccggagtgctggggctttgaactccgagaggaggtggaccagaacttttggaactagtgccggcggctctccaccccccagtataaaagaacgtgtggatcactttgctgagtacatccaagatttgaagaactgaaataaatcagctttaaacctgctttttaaaaatatctgggttggaatttgcccctgacaaataataaaatgatgagtgatgcaagtgacatgttggctgcagcgttggagcagatggatggtatcatagcaggttctaaggctctggaatattccaatgggatttttgattgccaatctcccacctctccattcatgggaagtttgcgagctctgcaccttgtggaagacctgcgtggattgttagagatgatggaaacagatgagaaagaaggcttgagatgccagatcccagattcaacagcagaaacgcttgttgaatggcttcagagtcaaatgacaaatggacacctaccagggaacggagatgtgtatcaagaaaggctggcacgtttagaaaatgataaagaatccctcgttcttcaggtaagtgtgttaacagaccaggtggaggctcagggagagaagattcgagatttggagttttgtcttgaagagcacagagagaaggtgaatgccacagaagaaatgctgcagcaggagcttctaagtaggacatccttagaaactcagaagttggatctgatggctgaaatatctaacttgaagttgaaactgacagctgtagagaaggacagattggattatgaagataagttcagagacacagaggggctgattcaggagatcaatgatttgaggttaaaagttagtgaaatggacagtgagagacttcagtatgaaaaaaagcttaaatcaaccaaagatgaactggcatctttaaaagaacaactagaagaaaaggaatctgaagtaaaaaggctacaagaaaaattggtttgcaagatgaaaggagaaggggttgaaattgttgatagagatgaaaattttaaaaagaagctcaaagaaaaaaacatcgaagtacaaaaaatgaaaaaagctgtggagtccttgatggcagcaaatgaagaaaaggatcggaaaatagaagatcttcgacagtgcctgaacaggtacaagaaaatgcaagacacggtggtactggcccaaggtaaaaaaggcaaagatggagaatatgaagagctgctcaattccagttccatctcctctttgctggatgcacagggtttcagtgatctggagaaaagtccatcacccactccagtaatgggatctcccagttgtgacccatttaacacaagtgttcccgaagagttccatactaccatcttgcaagtttccatcccttcattattgccagcaactgtaagcatggaaacttctgaaaaatcaaagttgactcctaagccagagacttcatttgaagaaaatgatggaaacataatccttggtgccactgttgatacccaactgtgtgataaacttttaacttcaagtctgcagaagtccagcagcctgggcaatctgaagaaagagacatctgatggggaaaaggaaactattcagaagacttcagaggacagagctccggcagaaagcaggccatttgggacccttcctcccaggcccccagggcaggacacctccatggatgacaaccccttcggcactcgaaaagtcagatcttcctttggccggggcttttttaaaatcaaaagtaacaagagaacagcaagtgcaccaaacttagatcgtaaacgaagtgccagtgcacccaccctagctgaaacagaaaaagagacagcagagcacctagatctggctggtgcttcttctcggccaaaagattcacagaggaacagtcccttccagataccgcctccatctccagattccaaaaagaaatccagaggtatcatgaaactctttggaaaacttaggagaagtcaatcaactacattcaacccagatgacatgtctgagcctgaattcaaaagaggagggacaagggcaaccgcggggccccgattaggttggtctcgagacttgggacagtctaacagtgacttggatatgccatttgccaagtggaccaaggagcaggtttgcaattggctgatggaacagggcttgggctcgtacctgaattctggcaagcactggattgcatctggccaaacgcttttgcaggcttctcaacaagatctagagaaggaacttggaatcaagcattcacttcatcgaaagaaactccagctagcactccaagccctgggatctgaagaagaaaccaatcatgggaagctggatttcaactgggtcactagatggttggatgacattggcctccctcaatataagacccagtttgatgaaggacgggttgatggtcgaatgctacattacatgactgttgatgacttactgtctctgaaggttgtaagtgtgctacaccatctcagtatcaaaagggccatccaggtcctgaggatcaataactttgaaccaaactgtctacggaggcggccatctgatgagaataccatcgccccatcagaagttcagaagtggactaaccatcgagtgatggagtggctgcgctccgtggacttggcagaatatgcgcccaatctcagaggcagtggtgtccatggtgggctcatggttctagagcctcgttttaacgtagaaacaatggctcagttattgaacatcccacccaataagactttgctgcgaagacatttggccactcatttcaaccttctgattggggctgaggcacagcaccagaagcgagatgccatggagctgccggattatgtacttctaacagctactgccaaagtgaagccaaagaaacttgcctttagcaattttgggaatttgagaaagaagaaacaggaagatggtgaagaatatgtttgtccaatggaattgggacaggcatcaggaagtgcatctaagaaaggatttaaacctggtttggatatgcgcctgtatgaggaagatgatttggaccggttagagcagatggaagattcagaagggacagtgagacagataggtgcattctctgaaggcatcaacaatctgacgcacatgttaaaagaagatgacatgtttaaagattttgctgcccgttcccccagtgccagcattacagatgaagactcaaacgtttgaccgtagcacctggatgaacattaggagtgcttagtcttttttctacttgcttttccaaacactcacagtatatacaacaggcagcggattgtctattgtttgttgttccaacttctgctgtcgagaagtttaaacagaaagcaggagtaatgtgccgattctgaagttgccacaaaaaataagacactggtgaatgagagtataattgtttttcttctatttaatgtaaaaatctgtgatatattatatttaaagtgttgcatttaagatgagtattttaccagagtgtttccattcatatccgcggtatggaggatttgaggaacagtaaccaggatgtgaatgattttgttacatcagtgttcactgtagccacctaagtaggacattatatgatttcagaatcaatatgtggaacttctttaagcattcagtgtgcccactaaatgccagccacacctccacttgcctcttattgtcttatttttatatatttttctaaatatatgtatatatacagtacatagaaaatagaacttttattttgtgacctaaggacgatggtgaaaagatcacgttttcaaaacaatctggtgatcagaatgttcatataccagctggtttctgaagaggtcagaatgatctttctccatactgacttttaacaatgttgatcattgaggctaaattaatatatatgaaatattcctttttgatgacaccacaaaattgttgaacagtttaagaatttcaaccttaatcttggatccctttacctcatatggaagaacttgagggacattagtatacttttttttaagatggagtcttgctctgtcacccaggttggagtgccatggcatgatcttggctcactgcaacctccacctcctgggtcaagccattctgcttcagccccaagtaggtgggactccaggcatgcaccaccatgcctggctaatttttgcatttttagtagagacagggtttcaccatattggccaggctgggactcgaactcctgaccttgtgatctgcccgcctcagcctcccaaagtactgggattataggcatgagccaccacgcccagcctgttatttttttattattattgtttttttttagtgacagagtctcattctgttgcccatgctggagtgcagtggcgtgatcatagctcactgcagccttgaattcctaggctccagtgatcctctcacctcagcttccctaatagctaggattacaggtgtgtggcctcccaccccaccccacccttcacacctggctgatttttcaaaaagtttttttgtagaaacagggtctcaccatgttgtccagcctggtctcaaactcctgtcctcaagtgatcctcccacctcagcctttcaaagtgctggaattacaggtgtgagccactctgcctggcctaccactaacttgaatacattcagaatcacctcctctccccaaaatttgtagaaatagtttttgaggaagccaaaagcaaagcagaaacctttacagtattgtttcttttctctttgttaactgtgtcattacagcaaaatactagcagtctgcctaaacatgttcattgtacatttctcaggctatcaatgaatggaggtttttaaaaagttgaatatttgtctgaacattttatttcaaagttcaaaaaaacagaggctgcaaaattcattttataatggctattttgtgacgataagatgtagttcatgtttttctgtagcactgggcccaaatattctttgtaaagaaaatcgctgcagcaaaaactgttactgtgtttattatatttgtagaagtattagaaaaatattctattttttattcagtgctgcgtaattacccatggtagccaaccctacaaaagacaggttttcacaaattgaggtggaggtgggcggttcagtatctgccactggacttgattataaactgtatttgaatatcagtggtattatcttttaagttgtcagcaagttaccaaggtattcattaaagaacttgtaatatcaaattactatttattcataacaattgatttgatgctaataataattttctttaaactctaccattcattatgtggtaactgtattgaacttactttatttggattttattttaatgtgactagatgtcaccacttcaaaaaatcaatttgttcttagaacctggttgaaaataccaggaaactgttacagacgccattttttttttttttgagacggagtcttgctctgttgcccaggctggagtgcagtggcacaatctcagctcactgcaagctccgcctcctgggttcacgccattctcccacctcagcttcccaagcagctgggactacaggtacctgccaccacgcctggctaattttgtttttgtatttttagtagagacggggtttcaccgtgttagccaggaaggtctcaatctcctgacctcgtgaatcgcccccctcggtctcccaaagtgctgggattacaggcgtgagccaccatgcccggcccaaatattttttattcaggatggtataacctaactgataataggtaataaggttaaatttttttatgacgtattttatttacaaatatcatacactgctggtgttaccatatgaaaggaaataaagtcaattgataattgcctc

SEQ ID NO: 4 - DNA, 4431 bp, Homo sapiens
gagtcgcggcgccatttgctgccgccgagcgtggacgcaggcggatctctgaagagctgggtcgccagcctctcccgcgcacgttgcctggcctccagcacctacttggtcccgcgcgctccctcgtgtcgcccctcggagcagcagccgccgcggtcgccgctacccggaaagaagtcagagacgccgcgaggtcgccgccaccgccatgcccaagaataaaggtaaaggaggtaaaaacagacgcaggggtaagaatgagaatgaatctgaaaaaagagaactggtattcaaagaggatggtcaggagtatgctcaggtaatcaaaatgttgggaaatggacggctagaagcaatgtgtttcgatggtgtaaagaggttatgtcacatcagaggaaaattgagaaaaaaggtttggataaatacctcggacattattttggttggtctccgagactaccaggataacaaagctgatgtaattttaaaatacaatgcagacgaagctagaagtctgaaggcatacggcgagcttccagagcatgctaaaatcaatgaaactgatacatttggtcctggagatgatgatgaaattcagtttgatgacattggagatgatgatgaagatattgatgacatctaaattgaactcaacattttacattccatcttttctgaagattgtcctacaatttggattttgatcatgacaaagaagattaaaatttcattagcatgaatgcaatttgttaaagcagactgatttgtttctaagatatttttggtttttttaaaactgataataatgctgaattatcttaagtgagatgttaagcccactttgttcttttaatgtaatggagcttatgggtagaagaccatgtctactaattacaaaaaaaaaaaaaaaccatgcattgctgcttttcctaccacttccagtaagaaaatgggtgttttgaagaaatcatttgccttgtcctcacggaatctgattaagccctggcctcttgattgtatagagtcattgtgtatattccagttacctagatattcccttgagattttgatacaatttgagggaggcagaagtctgcagttgaagaaaaaaaataagtctgtttgtcatatttaagtagcctgtggctatttttatactgattttgatatcatgttcttttcatagtcgtattttgccaccgtaaacataaaaaaaaaaaaaaagatttccaaaatgccgttttcagaacctgggttttaatagcagtattgaatttgtaagcttagtagttgcagaaattgaacactaggtggcactcagttatcttaacaggggaagtactgatacaattgttgacttttcttttactatgtgtaagaaataccccaaacatgaaaagattgttttgatcatatgcatgtatgtagaatatttttgcagagcagaaagattatgttagaagtgtgatttttattttcagaagtcatatacatgtaagctacaattttgagtgctttataaacacttaagatatatatataaattttaatttcatagcaacttgtaaaaaataaaatacttgttgaaaagcctttttcaacatatccctaagctaagggaagaggaaggaataacaactcagtgaaaagatggtctccagtttctgaatgaaaaagctacagctgagaaataaaataaaatgtcatgctgcagaatatgttatacccttattttgtgttaaggatatattttattatgtgaatggttttgtttttgttttttgtttttgttttttgcttgtattgggaattagctttactggtaacttccttatttagtttttagtggtcaactctaataaaatgaaactagggctgagctagttagccctcactagccaaactgaaactctatgcaacattaaaagaagagatccatcatgtagcttgtgacacttttattttattagtcaccggggaacttttcagtgatgaaaatacacagggtaataaaccttcacatggcttcaaaaggaaaacaagcaaatcttctctaatctactcttactataatttcctaagtgtacaccaaactctggatttaaaaatctgaagtactatagaacattaagttgaagaatggaaattaagagtacgtattcatggtttatatttcttattctatggagttcgtgaacacatctaggtggaatgcatctgagactaagggctggtttttaatcctcataagaaaccagccttgaagaattaacaattctcttcattggtattctaaacctcctaagatatttaggcttctgtacataaaagtgtttttgctaaatttacagtatatatagatcctttcatattattttactaagaatgtttgaactttgcatatttgatatagttcctggtaggaatagcacagctcaaacattagtttttctacttacctcctctaacacgtggtttgtctggagagtttctaaaaattcagctataaccccagttcatgtatttactggtgattgttcttgctgaggtagtaacagcccaatcttgggctgttaaatcctaggaaatctcgaatcatagtgattaaaatagttggggtaaagttgtagcttatatgcaatactacttggaggaattcttctactaatttgtatttaatgtggaaattgtatagtttcattgatttaatcataaataatggaaatggtctccaagaagttttatttttcatttttttgcttatacactctgattcctataatacagtgctataagctatgcacagaaaataaaatgtttgaaatccaagaataatggttcttactgctaagagggagtaatagttattactaatgattttgattgggttgcatttttgttgcaatgtttattccacttgcagttagaatatgaatatgttttatcactagtgtggctaaataaccaaacatttgtgtaaaaaaaaaaaaaagccaagatttcattgtttgttgaatatttcttaagcatctggcccctaaagagaccgcttcttaccaagcctgtaaactatgcatgatggaaattcttgtattttatttaggaatggctgttggtttactcaccacatctgtggaatcatggctataaatgtttgcttacaaactctttgtgacttgtaatttaacttaatctcatctaatgtaaatattagattatgatgttcagtaacatcttccataggtataaactgctgtcattattgatttcagagtaactctgagtaatcaaataggtaaaagcatgttttgagtaaaatagctagatttatactttacttgtatacagacttaacaacaaccggtattgactggattgacagctaaagtatcagaatgaaagcaaggtttttttgatgttacctgactgtcataaagatgaaaatgatttgtattggtatgaaatgcttatctttattctacttcgtaagggtaagttttatttatactctttggactcccatgaacttttgcacactgctttgtgtttttggtttaccctaaactaccatcctttttatctttgctttttttcttcctattcagaaaagagcaaaatgtgaaaagacacaagactctcaggtatagaatgaactgagcaatttggagaatgtattggactttgtcctctcttattcccccctcctagccctgcaagttgctaggtacttgtgaggcagtgtactggagaggggagagcatggatcctggggtcaaagggcctttgcccccacccttacttggccctctacctgcaggtgaccactggcacattctcctgcttgtctcagcttcaggttcttcacctctaagatggggatgatgaaaacagtacctgtcatgcagaattgttgggaggattgataatttagatgtttatacatgtaatgtacttagatcagtgtctgctcttttcacttgatatccagtactatgtaagatagaaggtgcatgtcttctgtattctgtatttcccatttcttttgcgtgcagtctttgattcgtacaatagaaggaacacgtagaatgtatatttgtacattcatgtcaacatagtatttgaaattgctaccaaactcatttaatttggcataagactaacagatgaagtctctcatttgcttgaagatattttacaaaataccaactgttctatatttctttagaaaaagattatagttattaatattgatacctctgataatattttattcttaaatcttcagtgattccttttactatagattcatgacagctaattagtactaactgatttagaggtttcctttcccatcatatggaatgatgtaaagaaatcagatacaaactactgcaattagaaaataaaatatgaacaactttcaacaatgta

SEQ ID NO: 5 - DNA, 2065 bp, Homo sapiens
gcagtcgctgccgaccggctggctgggccttgcggcgtgaggaccccggcggcgccgcagtcccgcgagccatggcccagtccggcggggaggctcggcccgggcccaagacggcggtgcagatccgcgtcgccatccaggaggccgaggacgtggacgagttggaggacgaggaggagggggcggagactcggggcgccggggacccggcccggtacctcagccccggctggggcagcgcgagcgaggaggagccgagccgcgggcacagtggcaccactgcaagtggaggtgagaacgagcgtgaggacctggagcaggagtggaagcccccggatgaggagttgatcaagaaactggtggatcagatcgaattctacttttctgatgaaaacctggagaaggacgcctttttgctaaaacacgtgaggaggaacaagctgggatatgtgagcgttaagctactcacatccttcaaaaaggtgaaacatcttacacgggactggagaaccacagcacatgctttgaagtattcagtggtccttgagttgaatgaggaccaccggaaggtgaggaggaccacccccgtcccactgttccccaacgagaacctccccagcaagatgctcctggtctatgatctctacttgtctcctaagctgtgggctctggccaccccccagaagaatggaagggtgcaagagaaggtgatggaacacctgctcaagctttttgggacttttggagtcatctcatcagtgcggatcctcaaacctgggagagagctgccccctgacatccggaggatcagcagccgctacagccaagtggggacccaggagtgcgccatcgtggagttcgaggaggtggaagcagccatcaaagcccatgagttcatgatcacagaatctcagggcaaagagaacatgaaagctgtcctgattggtatgaagccacccaaaaagaaacctgccaaagacaaaaatcatgacgaggagcccactgcgagcatccacctgaacaagtccctgaacaagagagtcgaggagcttcagtacatgggtgatgagtcttctgccaacagctcctctgaccccgagagcaaccccacatcccctatggcgggccgacggcacgcggccaccaacaagctcagcccgtctggccaccagaatctctttctgagtccaaatgcctccccgtgcacaagtccttggagcagccccttggcccaacgcaaaggcgtttccagaaagtccccactggcggaggaaggtagactgaactgcagcaccagccctgagatcttccgcaagtgtatggattattcctctgacagcagcgtcactccctctggcagcccctgggtccggaggcgtcgccaagccgagatggggacccaggagaaaagccccggtacgagtcccctgctctcccggaagatgcagactgcagatgggctacccgtaggggtgctgaggttgcccaggggtcctgacaacaccagaggatttcatggccatgagaggagcagggcctgtgtataaataccttctatttttaatacaagctccactgaaaaccaccttcgttttcaaggttctgacaaacacctggcatgacagaatggaattcgttcccctttgagagattttttattcatgtagacctcttaatttatctatctgtaatatacataaatcggtacgccatggtttgaagaccaccttctagttcaggactcctgttcttcccagcatggccactattttgatgatggctgatgtgtgtgagtgtgatggccctgaagggctgtaggacggaggttccctgggggaagtctgttctttggtatggaatttttctctcttctttggtatggaatttttcccttcagtgactgagctgtcctcgataggccatgcaagggcttcctgagagttcaggaaagttctcttgtgcaacagcaagtagctaagcctatagcatggtgtcttgtaggaccaaatcgatgttacctgtcaagtaaataaataataaaacacccaaaaaaaaaaaaaaaaaa

SEQ ID NO: 6 - DNA, 2820 bp, Homo sapiens
cgggaggctgcgaggactgcaaaagggtggagtcggcctcgcccccgcccaggccccgcccctgccgggaacccactttcccagtcctaggcggcggtcagatccttgcaagcatggtcgcgccggggcttgtactcgggctggtgctgccattaatcctgtgggccgacagaagtgcaggtattggttttcgctttgcttcatacatcaataatgatatggtgctgcagaaggagcctgctggggcagtgatatggggcttcggtacacctggagccacagtgaccgtgaccctgcgccaaggtcaggaaaccatcatgaagaaagtgaccagtgtgaaagctcactctgatacgtggatggtggtactggatcctatgaagcctggaggacctttcgaagtgatggcacaacagactttggagaaaataaacttcaccctgagagttcatgacgtcctgtttggagatgtctggctctgtagtgggcagagtaacatgcagatgactgtgttacagatatttaatgctacaagggagttgtctaacactgcggcatatcagtctgtccgcatcctctctgtctctcccattcaagcagagcaggagctggaggaccttgttgcggttgacttgcagtggtctaagcccacctcagaaaacttaggccatggatatttcaagtacatgtcagcagtgtgctggctctttggacgtcacctttatgacactctgcagtatcccatcgggctgatcgcctccagctggggcgggacacccattgaagcctggtcatctggacggtcactgaaagcctgtggggtccctaaacaagggtccattccatacgattctgtaactggtcccagtaagcactctgttctctggaatgccatgatccatccactgtgcaatatgactctgaaaggggtagtatggtaccagggggagtccaatataaattataacacggatctgtacaattgcacattccctgcactcatcgaagactggcgtgaaaccttccaccgtggttcccaggggcagacggagcgtttcttcccatttggacttgtccagttatcttcagatttgtctaagaagagctcagacgatggatttccccagatccgttggcatcaaacagcagacttcggctatgtccccaacccaaagatgcccaatactttcatggctgtagctatggatctctgtgatagagactcgccttttggcagcatccaccctcgagataaacagactgtggcttatcggctgcatttgggggcccgtgctctggcttatggtgagaagaatttgacctttgaaggaccactgcctgagaagatagaactcttggctcacaaggggctgctcaatctcacatattaccagcaaatccaggtgcagaaaaaggacaacaagatatttgagatctcctgttgcagtgaccatcgatgcaagtggcttccagcttctatgaacaccgtctccacccagtccctgaccctggcgatcgattcttgtcatggcactgtggttgctctccgctatgcttggaccacgtggccttgtgaatataagcagtgtcccctataccaccccagtagtgccctgccagcccctcccttcattgctttcattacagaccagggtcctggacatcagagcaatgttgctaaatgactgtttcagtatgatcagaacttagatataaggatgggtccttcagattttagcatttaggagtttcaataataaccattgcttttaaaggaaattaatagaaagcctcattgaatggctttcagctagcacatggctgtttctatattctgatgagcccaggcttataggtaacttgaaatgcttgctttttgttccctagttggtctaagggtctgtattggactaattctgaactacagacaaattggacctcaatgtcatttacttccctcatattaatgggagtgaaatgtctaatacttttgcccctttttatccagagttgtgggaatctcaggattggaagagattttaaaggccacataggccagctagtgttcatgtgttctttataaaatttctcccatccaagtactaaccaggcccgaccctgcttagcttccgagatcagatgagatcaggcgcgttcagggtgatatggccgtagacgtctttacaaaattcctgacaggtggttactgaatctctctatgaactttccattcaaaactttccaagtttttccttatgtggaaccgaaatctttctttctcccgtgaaactttactactatcagataattgaagacagatctctttgtattctcttcaagcccaaaccaattctgttccttcaatctaaatagtggtaatatgaatgtttaagaaatgaaataagaaacatgtgcaggcactttggaaggtgctaagtgactgccctaaggaatgaaaagcaagggccaggtgggagtagcccagcgaaggcacttgggctgccaggaacaggaggcgtgggaaactctggcttaggaaaacatgaacacaggggcaacagaggcaaactgttgttcgagttaaatataaatctcaggctctttaaaggtaaaaggtttaaggataatccatttggaagaagaaaagagtgaggctgaaagtaaagccacatgacaagcatataaaaaaaaatgcagatgatacaaatatgaaagaggccttcagtgtttgtttattaagaatcttaatgcagtttactgatggattaaaaacagctaacattgtctgaaaattatgttacctataagaagttggaaata

SEQ ID NO: 7 - DNA, 681 bp, Homo sapiens
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgatagccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataa

SEQ ID NO: 8 - DNA, 346 bp, Homo sapiens
ataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgagccgaat

SEQ ID NO: 9 - DNA, 3843 bp, Homo sapiens
gagcgagagtgtgtcgagtgagtgtgcgtctgtgtgtcccggcgagggtgcgcgctcggcgccgggagcgcggccagccgagtccggaggcatcgggaggtcgagagccgccgggaccccagctctgcgttcactgccccgtccggagctggacttcggggccggggccggggccgtgcgccggggacaggcagggccgggtcgcgggccgcgcgtcccccaggccggagatctgcgagtgaagagggacgagggaaaagaaacaaagccacagacgcaacttgagactcccgcatcccaaaagaagcaccagatcagcaaaaaaagaagatgggccccccgagcctcgtgctgtgcttgctgtccgcaactgtgttctccctgctgggtggaagctcggccttcctgtcgcaccaccgcctgaaaggcaggtttcagagggaccgcaggaacatccgccccaacatcatcctggtgctgacggacgaccaggatgtggagctgggttccatgcaggtgatgaacaagacccggcgcatcatggagcagggcggggcgcacttcatcaacgccttcgtgaccacacccatgtgctgcccctcacgctcctccatcctcactggcaagtacgtccacaaccacaacacctacaccaacaatgagaactgctcctcgccctcctggcaggcacagcacgagagccgcacctttgccgtgtacctcaatagcactggctaccggacagctttcttcgggaagtatcttaatgaatacaacggctcctacgtgccacccggctggaaggagtgggtcggactccttaaaaactcccgcttttataactacacgctgtgtcggaacggggtgaaagagaagcacggctccgactactccaaggattacctcacagacctcatcaccaatgacagcgtgagcttcttccgcacgtccaagaagatgtacccgcacaggccagtcctcatggtcatcagccatgcagccccccacggccctgaggattcagccccacaatattcacgcctcttcccaaacgcatctcagcacatcacgccgagctacaactacgcgcccaacccggacaaacactggatcatgcgctacacggggcccatgaagcccatccacatggaattcaccaacatgctccagcggaagcgcttgcagaccctcatgtcggtggacgactccatggagacgatttacaacatgctggttgagacgggcgagctggacaacacgtacatcgtatacaccgccgaccacggttaccacatcggccagtttggcctggtgaaagggaaatccatgccatatgagtttgacatcagggtcccgttctacgtgaggggccccaacgtggaagccggctgtctgaatccccacatcgtcctcaacattgacctggcccccaccatcctggacattgcaggcctggacatacctgcggatatggacgggaaatccatcctcaagctgctggacacggagcggccggtgaatcggtttcacttgaaaaagaagatgagggtctggcgggactccttcttggtggagagaggcaagctgctacacaagagagacaatgacaaggtggacgcccaggaggagaactttctgcccaagtaccagcgtgtgaaggacctgtgtcagcgtgctgagtaccagacggcgtgtgagcagctgggacagaagtggcagtgtgtggaggacgccacggggaagctgaagctgcataagtgcaagggccccatgcggctgggcggcagcagagccctctccaacctcgtgcccaagtactacgggcagggcagcgaggcctgcacctgtgacagcggggactacaagctcagcctggccggacgccggaaaaaactcttcaagaagaagtacaaggccagctatgtccgcagtcgctccatccgctcagtggccatcgaggtggacggcagggtgtaccacgtaggcctgggtgatgccgcccagccccgaaacctcaccaagcggcactggccaggggcccctgaggaccaagatgacaaggatggtggggacttcagtggcactggaggccttcccgactactcagccgccaaccccattaaagtgacacatcggtgctacatcctagagaacgacacagtccagtgtgacctggacctgtacaagtccctgcaggcctggaaagaccacaagctgcacatcgaccacgagattgaaaccctgcagaacaaaattaagaacctgagggaagtccgaggtcacctgaagaaaaagcggccagaagaatgtgactgtcacaaaatcagctaccacacccagcacaaaggccgcctcaagcacagaggctccagtctgcatcctttcaggaagggcctgcaagagaaggacaaggtgtggctgttgcgggagcagaagcgcaagaagaaactccgcaagctgctcaagcgcctgcagaacaacgacacgtgcagcatgccaggcctcacgtgcttcacccacgacaaccagcactggcagacggcgcctttctggacactggggcctttctgtgcctgcaccagcgccaacaataacacgtactggtgcatgaggaccatcaatgagactcacaatttcctcttctgtgaatttgcaactggcttcctagagtactttgatctcaacacagacccctaccagctgatgaatgcagtgaacacactggacagggatgtcctcaaccagctacacgtacagctcatggagctgaggagctgcaagggttacaagcagtgtaacccccggactcgaaacatggacctgggacttaaagatggaggaagctatgagcaatacaggggacaactgtgggaaggctgggaaggttaagaaacaacagaggtggacctccaaaaacatagaggcatcacctgactgcacaggcaatgaaaaaccatgtgggtgatttccagcagacctgtggtattggccaggaggcctgagaaagcaagcacgcactctcagtcaacatgacagattctggaggataaccagcaggagcagagataacttcaggaagtccatttttgcccctgcttttgctttggattatacctcaccagctgcacaaaatgcattttttcgtatcaaaaagtcaccactaaccctcccccagaagctcacaaaggaaaacggagagagcgagcgagagagatttccttggaaatttctcccaagggcgaaagtcattggaatttttaaatcataggggaaaagcagtcctgttctaaatcctcttattcttttggtttgtcacaaagaaggaactaagaagcaggacagaggcaacgtggagaggctgaaaacagtgcagagacgtttgacaatgagtcagtagcacaaaagagatgacatttacctagcactataaaccctggttgcctctgaagaaactgccttcattgtatatatgtgactatttacatgtaatcaacatgggaacttttaggggaacctaataagaaatcccaattttcaggagtggtggtgtcaataaacgctctgtggccagtgtaaaagaaaatccctcgcagttgtggacatttctgttcctgtccagataccatttctcctagtatttctttgttatgtcccagaactgatgttttttttttaaggtactgaaaagaaatgaagttgatgtatgtcccaagttttgatgaaactgtatttgtaaaaaaaattttgtagtttaagtattgtcatacagtgttcaaaaccccagccaatgaccagcagttggtatgaagaacctttgacattttgtaaaaggccatttcttgg

SEQ ID NO: 10 - DNA, 1855 bp, Homo sapiens
acccaaagtcttcaagcctggagttcctgcttggttcttcctgaggactgagcaccttctagactacatccagatctgttttccctgcagattcgtgaagatgagcatccggactccacccagactcctggagcttgcagggcggagcctgctgagggaccaagccttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggctggatgcactgcttacccaaggggttcatcccaggaggtggaaacttcaagtgctggatttacaggatgtctgtgagaacttctggatggtttggtctgaagctatggcccatgggtgcttcctcaatgccaagaggaacaaaaaaccagtgcaggactgtccaaggatgagaggacagcagcccttgactgtgttcgtagaactttggctcaagaacaggactctggatgaatacctcacctgcctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgtgctgcctccaaaagctttctatgaactctgtttctttcctcgaaggccacctggaccagctgctcagctgtctgaagacctcgttaaaggtcctcacaataactaactgtgtgcttttggaatcagacttgaagcatctatcccagtgcccgagtatcagtcaactaaagaccctggacctgagtggcatcagactgaccaattacagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctccatggccaccctggagaacctgctgagccacacaatcatactcaaaaacttatgcgtggagctgtatcctgccccccgggagagttatgatgctgatggtactctctgctggagcagatttgctcaaattagggctgagctgatgaagagagtgagggacttaaggcaccccaagaggatcttgttctgtactgactgctgccctgactgtggcaacaggtcattttatgacctggaggcagatcaatgctgctgttgaatgcctgcctatttgggtggatatgtcaaacgctttcttctggacacttggaaactaaaacctaggtcttaggtacatcctatagggagcacagaacccatcatttcacacatgggctctgaaagtgggaaaggaaaggtgatcaagcaggggcaggacttgggggaagtgttgccatggattcgatgggactttggggacctgtgtcctgtagagtggaaaatgggaatttgaatgtctagagtggaggcttgagaatacttgagggagttactcttggatgcatggttgtaaagaaacaatcagaaataaaggaaaactgag

SEQ ID NO: 11 - DNA, 2123 bp, Homo sapiens
ctgataagtttgtcttttctctggatttttcttgcagatttatcaggatgagcttccaggccccacgcagactcctggagctggcagggcagagcctgctgagggaccaggccttggccatctccgtcctggatgagctgcccagggagctcttcccccgactgttcgtggaggccttcactagcagacgctgcgaggttctgaaggtgatggtgcaggcctggcccttcccctgcctccctctggggtccctgatgaagacgcctgatctggagatcttacattatgtagtggatgggattgattgcctgcttgcccaaaaggttcgccccaggaggtggaaacttcaagtgctggaaatgcgggatgttgatgagaatttttggaccatatggtctggagccaggcccctgtcctgctccccagaggccatgagtaagagacagacagtggaggactgtccaaggacaggagagaagcagcccttgaaggtgttcatggatgtttgcctcaaggaaaaatccgtggatgaagatctgagcttcttctctgggtgggtgcagcacagaagacgttcagtacacctgtgctgtactaaggtggtaaattattcaatgaacattctaaatttcagaaacatattagaaacagtatacccagacagtatccaagtattggaaatttggaacatgtgctggccgtgtatggtagcagaggttagccgttacctgagccagatgaagaatcttcgaaaactcttcatctccgatggctgtggttacctgccaagctttgagagccaaggacagttagttgctgaattcagctctgtgttcctcaggctggagtacctccagatgctttatatgagaaggatccgcttcttcgaaggctacctggaccagctgatcaggtgcctcaagagcccgttggagacattggcattaacttatggctccctagatgaagaggacttgaaatgtctgccctggtacccaagtctcagtcaactgaagcagctgaatctgagtcatggtacactgcgcttcatccgtcttgagcccctccgagctctgctagagaaagttgctgccactcttcagaccctcttcttagtggactgtgggattggggactccaaactcagggtcatcctgcctgccctgagccgctgctccaacctcaccactttctgctttcacggcaatgacacgtccatggatggtctgaaggacctgctgcgccacacaggcaggctgagcaatttgagcctggaaacatatcctgcccctcgggagagtcttgacaacaggggtcgtgtcatttcggagctcctcaccccacttcaggctgagctgatgcgtatactgagggaagtaagggagcccaacaggatcttctttggtcccgtctcctgcccttgctgtggcatgtcacccactgagcaactggagttcaatttttgcttgcggggaaggcctgcctagtggggtggaggtataaaaagctttttctccaggcacttggaaactaaaatctaggacatagatatcttttatttttctttttccttattttacaattttacagcttttatttaaaaatttgagacagggtttccctatgttgtccaggctggtctcaaactcttacgcttaagggagccccctgcttggcctcccaagattctgggattacaggcataagcagctgtgccgggtctataggtgtattataaagggagcagagaaacctctgtttcaggcatgtgctttctgtgagtgggaaaaaaaacacaaaaaaacccagcagggggcagcactggggaaaaggttgaatggagtcactgagactcagggatctgtgtcctagacagtcagaaatagaacctgaagttctagagtgaaggagttatctcagcaaggatggatacaaagaaacgtcggaagtaaagggaacctaaatggaaactctctgctgtccttcatgattgattagcctgtttcagcaatttatacatcagaaatctttagttcctgatgaattaaaaaaagaggtactagttcatctgtgatttaagttcatccgcaggaaataaaggaatcaaaataaacttcattttg

SEQ ID NO: 12 - DNA, 1863 bp, Homo sapiens
acccaaagtcttcaagcctggagttcctgcttggttcttcctgaggtctgagcaccttctagactacatccagatctgttttccctgcagattcatgaagatgagcatccggactccacccagactcctggagcttgcagggcggagcctgctgagggaccaagctttggccatgtccaccctggaggagctgcccacagaacttttccccccactgttcatggaggccttcagcaggagacgctgtgaggccctgaagctgatggtgcaggcctggcccttccgccgcctccctctgaggcctctgataaagatgccttgtctggaggccttccaagctgtgctcgatgggcttgatgcactgcttacccaaggggttcgtcccaggaggtggaaactccaagtgctggatttacaggatgtctgtgagaacttctggatggtttggtctgaagctatggcccatgggtgcttcctcaatgccaagaggaacaaaaaaccagtgcaggactgtccaaggatgagaggacggcagcccttgactgtgttcgtagaactttggctcaagaacaggactctggatgaatacctcacctacctccttctatgggtcaagcagaggaaagatttactacacctgtgctgtaagaagctgaaaattttgggaatgcccttccgcaatatcagaagcatcctgaaaatggtgaacctagactgtatccaggaggtggaagtgaattgcaagtgggtactgcccatcctgacacagtttaccccatacctgggccacatgaggaatcttcagaagctcgttctctcccacatggatgtctctcgctacgtttccccagagcagaagaaggagattgttacccagttcaccactcagttcctcaagctgcgctgcctccaaaagctttatatgaactctgtttctttcctcgaaggccacctggaccagctgctcagctgtctgaagacctcgttaaaagtcctcacaataactaactgtgtgcttttggaatcagacttgaagcatctatcccagtgcccgagtatcagtcaactaaagaccctggacctgagtggcatcagactgaccaattatagtcttgtgcctctccaaattctcctagaaaaagttgcagccacccttgagtacctggatttagatgactgtggcatcatagactcccaagtcaacgccatcctgcctgccctgagccgctgctttgagctcaacaccttcagcttctgtggaaatcccatctgcatggccaccctggagaacctgctgagccacacaatcatactcaaaaacttatgtgtggagctgtatcctgccccccgagagagttatggtgctgatggtactctctgctggagcagatttgctcaaattagggctgagctgatgaacagagtgagggacttaaggcaccccaagaggatcttgttctgtactgactactgccctgactgtggcaacaggtcattttatgacctggaggcagatcaatactgctgttgaatgcctgcctatttggatgggtatgtcaaacgctttcttctggacacttggaaactaaaacctaggtcttaggtacatcctaaagggagcacagaacccatcatttcacacataggctctgaaagtgggaaaggaaagctgatcaagcaggggccggacttgggggaaatgttgccatggattcgatgggactttggggacctgtgtcctgtagattcgaaaatgggaatctgaatgtctagagtggaattcaggcttgagaatacatgagggagttactcttgcatggatggttgtaaagaaacaatcagaaataaaggaaaactgagcag

SEQ ID NO: 13 - DNA, 2123 bp, Homo sapiens; identical to SEQ ID NO: 11

SEQ ID NO: 14 - DNA, 2123 bp, Homo sapiens
ctgataagtttgtcttttctctggatttttcttgcagatttatcaggatgagcttccaggccccacgcagactcctggagctggcagggcagagcctgctgagggaccaggccttggccatctccgtcctggatgagctgcccagggagctcttcccccgactgttcgtggaggccttcactagcagacgctgcgaggttctgaaggtgatggtgcaggcctggcccttcccctgcctccctctggggtccctgatgaagacgcctgatctggagatcttacattatgtagtggatgggattgattgcctgcttgcccaaaaggttcgccccaggaggtggaaacttcaagtgctggaaatgcgggatgttgatgagaatttttggaccatatggtctggagccaggcccctgtcctgctccccagaggccatgagtaagagacagacagtggaggactgtccaaggacaggagagaagcagcccttgaaggtgttcatggatgtttgcctcaaggaaaaatccgtggatgaagatctgagcttcttctctgggtgggtgcagcacagaagacgttcagtacacctgtgctgtactaaggtggtaaattattcaatgaacattctaaatttcagaaacatattagaaacagtatacccagacagtatccaagtattggaaatttggaacatgtgctggccgtgtatggtagcagaggttagccgttacctgagccagatgaagaatcttcgaaaactcttcatctccgatggctgtggttacctgccaagctttgagagccaaggacagttagttgctgaattcagctctgtgttcctcaggctggagtacctccagatgctttatatgagaaggatccgcttcttcgaaggctacctggaccagctgatcaggtgcctcaagagcccgttggagacattggcattaacttatggctccctagatgaagaggacttgaaatgtctgccctggtacccaagtctcagtcaactgaagcagctgaatctgagtcatggtacactgcgcttcatccgtcttgagcccctccgagctctgctagagaaagttgctgccactcttcagaccctcttcttagtggactgtgggattgggtactccaaactcagggtcatcctgcctgccctgagccgctgctccaacctcaccactttctgctttcacggcaatgacacgtccatggatggtctgaaggacctgctgcgccacacaggcaggctgagcaatttgagcctggaaacatatcctgcccctcgggagagtcttgacaacaggggtcgtgtcatttcggagctcctcaccccacttcaggctgagctgatgcgtatactgagggaagtaagggagcccaacaggatcttctttggtcccgtctcctgcccttgctgtggcatgtcacccactgagcaactggagttcaatttttgcttgcggggaaggcctgcctagtggggtggaggtataaaaagctttttctccaggcacttggaaactaaaatctaggacatagatatcttttatttttctttttccttattttacaattttacagcttttatttaaaaatttgagacagggtttccctatgttgtccaggctggtctcaaactcttacgcttaagggagccccctgcttggcctcccaagattctgggattacaggcataagcagctgtgccgggtctataggtgtattataaagggagcagagaaacctctgtttcaggcatgtgctttctgtgagtgggaaaaaaaacacaaaaaaacccagcagggggcagcactggggaaaaggttgaatggagtcactgagactcagggatctgtgtcctagacagtcagaaatagaacctgaagttctagagtgaaggagttatctcagcaaggatggatacaaagaaacgtcggaagtaaagggaacctaaatggaaactctctgctgtccttcatgattgattagcctgtttcagcaatttatacatcagaaatctttagttcctgatgaattaaaaaaagaggtactagttcatctgtgatttaagttcatccgcaggaaataaaggaatcaaaataaacttcattttg

SEQ ID NO: 15 - DNA, 1589 bp, Homo sapiens
ccgtcgcccggatcccctgagctgcccgccatcccacgtgaccgcgccgccccccagctccaccgctgagcccgctcgccatggccctcttcggggccctcttcctagcgctgctggcaggcgcacatgcagagttcccaggctgcaagatccgcgtcacctccaaggcgctggagctggtgaagcaggaggggctgcgctttctggagcaagagctggagactatcaccattccggacctgcggggcaaagaaggccacttctactacaacatctctgaggtgaaggtcacagagctgcaactgacatcttccgagctcgatttccagccacagcaggagctgatgcttcaaatcaccaatgcctccttggggctgcgcttccggagacagctgctctactggttcttgaaggtgtatgattttctctccacgttcatcacctcagggatgcgcttcctcctcaaccagcagatctgccctgtcctctaccacgcagggacggtcctgctcaactccctcctggacaccgtgcctgtgcgcagttctgtggacgagcttgttggcattgactattccctcatgaaggatcctgtggcttccaccagcaacctggacatggacttccggggggccttcttccccctgactgagaggaactggagcctccccaaccgggcagtggagccccagctgcaggaggaagagcggatggtgtatgtggccttctctgagttcttcttcgactctgccatggagagctacttccgggcgggggccctgcagctgttgctggtgggggacaaggtgccccacgacctggacatgctgctgagggccacctactttgggagcattgtcctgctgagcccagcagtgattgactccccattgaagctggagctgcgggtcctggccccaccgcgctgcaccatcaagccctctggcaccaccatctctgtcactgctagcgtcaccattgccctggtcccaccagaccagcctgaggtccagctgtccagcatgactatggacgcccgtctcagcgccaagatggctctccgggggaaggccctgcgcacgcagctggacctgcgcaggttccgaatctattccaaccattctgcactggagtcgctggctctgatcccattacaggcccctctgaagaccatgctgcagattggggtgatgcccatgctcaatgagcggacctggcgtggggtgcagatcccactacctgagggcatcaactttgtgcatgaggtggtgacgaaccatgcgggattcctcaccatcggggctgatctccactttgccaaagggctgcgagaggtgattgagaagaaccggcctgctgatgtcagggcgtccactgcccccacaccgtccacagcagctgtctgagccctcaatccccaagctggcagctgtcattcaggaccccaacccctctcagcccctcttttcccacattcatagcctgtagtgccccctctaacccccagtgccacagagaagacgggatttgaagctgtacccaatttaattccataatcaatctatcaattacagtccgtccaccacctc

SEQ ID NO: 16 - DNA, 3778 bp, Homo sapiens
tttttaaattttgcatttgacttaaagtgccatgagaaaatttgcatactgcaaggtggtcctagccacctccttgatttgggtactcttggatatgttcctgctgctttacttcagtgaatgcaacaaatgtgatgaaaaaaaggagagaggacttcctgctggagatgttctagagccagtacaaaagcctcatgaaggtcctggagaaatggggaaaccagtcgtcattcctaaagaggatcaagaaaagatgaaagagatgtttaaaatcaatcagttcaatttaatggcaagtgagatgattgcactcaacagatctttaccagatgttaggttagaagggtgtaaaacaaaggtgtatccagataatcttcctacaacaagtgtggtgattgttttccacaatgaggcttggagcacacttctgcgaactgtccatagtgtcattaatcgctcaccaagacacatgatagaagaaattgttctagtagatgatgccagtgaaagagactttttgaaaaggcctttagagagttatgtgaaaaaactaaaagtaccagttcatgtaattcgaatggaacaacgttctggattgatcagagctagattaaaaggagctgctgtgtctaaaggccaagtgatcaccttcctggatgcccattgtgagtgtacagtgggatggctggagcctctcttggccaggatcaaacatgacaggagaacagtggtgtgtcccatcatcgatgtgatcagtgatgatacttttgagtacatggcaggctctgatatgacctatggtgggttcaactggaagctcaattttcgctggtatcctgttccccaaagagaaatggacagaaggaaaggtgatcggactcttcctgtcaggacacctaccatggcaggaggccttttttcaatagacagagattactttcaggaaattggaacatatgatgctggaatggatatttggggaggagaaaacctagaaatttcctttaggatttggcagtgtggaggaactttggaaattgttacatgctcacatgttggacatgtgtttcggaaagctacaccttacacgtttccaggaggcacagggcagattatcaataaaaataacagacgacttgcagaagtgtggatggatgaattcaagaatttcttctatataatttctccaggtgttacaaaggtagattatggagatatatcgtcaagagttggtctaagacacaaactacaatgcaaacctttttcctggtacctagagaatatatatcctgattctcaaattccacgtcactatttctcattgggagagatacgaaatgtggaaacgaatcagtgtctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcaccctgccagaaatattctgagaccaaatttacaaaaaaacgaaaaaaataaggattgactgggctacctcagcatacatttctgccacattcttaagtagcaaaaaaggaaaagtgctttcctcctctgcaggatgtaaggtttatcagccattaaaacttagacttctctagcttttcactagctgtgaaccagccttcctgtccatggacgtgaaactgcatagtaatgagactgtgcacactgatgtttacaagattgaaagagtctttctccgaaaatcatggtaaagaatactgagacaatgaaaaaaaatcaacaaaatatgctttctggagaactgtaccttctatggtttgcttgcacatcagtagtttctgctgaacgtgctgtcataatgaagagatttccaagattttttttcctgattagaacgggtagccagtatattaaatattgatagaaaaataaaagaactggaaccagattcagaatcttgaaaacaacattttttacaacaaacaaaaaaactatattaaacagggtttaaaggaaaattaaaacagaactatgaagaagtacaatttgttatagtatagtatcaaatttctatatagattttatacctcagtggggaaaaataactgattccaatgacattcattttgttttcatctgtgatagtcatggatgcttttattttccttggggtgctgaaattgagctgaaaaaaaaaggctctttgaatatagttttaatttctctctacagttttttttgtttggtttgtgggctgttggaattgtaatttttaattgccttctaaaaaatggaaatttaacaatgtctgatctcagctgaacaaattagatgtttcagttgctcttgggtcaactggcttacagatttacatgtgcacacacacacaaatttcttatcacattttcgacttcttcacttgacctaactgattatgcgaaatacccaagattcatgctactgttccacatttgttttcacagcaataaatcttcagttctgttgtttatgattccacttaacaaggggcctgcaaatgtgatttattatttgggtatttggagataatacatttgagggttttttggaaaacctttttcactccatactcaaatatgcttcattgtcaaatgcatatttaaattaaattattgaattgtaatgtttatctgctgctttttttaaataaaatttgactgaaaatgtttaattggcattttttaatgacttacccaagaaaagtgcagctattattccatattaataggcttgcatttcttttcctaaatcttatttaggctaaatcagttttattgtcctctgattttttttaataccacagaaatcacctgagtgtcaattgaaaagttgtcaattaaaaggtaaccttttaactctcgtaggaggaatctcattaagacatttttcctgatatgtagagcagtctgttggcaaaaatgcatatattttctttcatatttgtaaaattatatttaatggaattcttttctttgattatcaaggactttcactgcaggcagtgctatttcttgtgcctaagaatgtttccaaaagtcgcatcgctaatgatatttgccaagttgagtgtacacaaagtttctcatatcctgttcaagttaatcaacatcaaacacatggggatgctttagggtgagtctataatacaaaatgcataaaccatgtccccaggaaatttgaaaggaagcaagtgctgaatggaatttttttccttttccatgagctgtgttaattctatctccagtaggcctaatgcttgaaataagcaagatgtctaatcaataaattattttcatgctcagaatttcaggtttttgtactccagcatagcttggtcttatttcttactgtatgaaagcttaacagcaatgtgatttaaggttttgttttaaatgggagatgtaagtgatttaattcatgggtacttttagaacctgatagataatcccattgcctttatttttctaattaaagaatcctaaatactttgaaaatacaaaatattcctg

SEQ ID NO: 17 - RNA, 19 nt, Artificial
  siRNA
ccucaacuguucacaacau

SEQ ID NO: 18 - RNA, 19 nt, Artificial
  siRNA
gagauacacagucggaaau

SEQ ID NO: 19 - RNA, 19 nt, Artificial
  siRNA
cuggcuuccuagaguacuu

SEQ ID NO: 20 - RNA, 19 nt, Artificial
  siRNA
gaggcaagcugcuacacaa

SEQ ID NO: 21 - RNA, 19 nt, Artificial
  siRNA
gacacaugauagaagaaau

SEQ ID NO: 22 - RNA, 19 nt, Artificial
  siRNA
gagauuacuuucaggaaau

SEQ ID NO: 23 - DNA, 70 bp, Homo sapiens
tcccaaggtacagtggaccaacatctccagttccaaaaaccctcacaccaggtgcacttctccaaaacag

SEQ ID NO: 24 - DNA, 70 bp, Homo sapiens
gtgaaaaaatgatgaagacgggtgcacctgtctgagtttggccctcatgtgagctgtgcccttccctctc

SEQ ID NO: 25 - DNA, 70 bp, Homo sapiens
ggcgcgttcagggtgatatggccgtagacgtctttacaaaattcctgacaggtggttactgaatctctct

SEQ ID NO: 26 - DNA, 70 bp, Homo sapiens
aactcagaagttggatctgatggctgaaatatctaacttgaagttgaaactgacagctgtagagaaggac

SEQ ID NO: 27 - DNA, 70 bp, Homo sapiens
atgagcgggcgcagtgattataggctttcgctctaagattaaaaatgccctagcccacttcttaccacaa

SEQ ID NO: 28 - DNA, 69 bp, Homo sapiens
ttttcaggagtggtggtgtcaataaacgctctgtggccagtgtaaaagaaaatccctcgcagttgtgga

SEQ ID NO: 29 - DNA, 70 bp, Homo sapiens
tttaaaatacaatgcagacgaagctagaagtctgcaggcatacggcgagcttccagagcatgctaaaatc

SEQ ID NO: 30 - DNA, 70 bp, Homo sapiens
tgatgaagacgcctgatctggagatcttacattatgtagtggatgggattgattgcctgcttgcccaaaa

SEQ ID NO: 31 - DNA, 70 bp, Homo sapiens
agctgccttatcttattcctttttgtatttccatacctagcctaatacctagcttatataagggaacaga

SEQ ID NO: 32 - DNA, 70 bp, Homo sapiens
cccgcccgcgtccctttctccataaaattcttcttagtagctattaccttcttattatttgatctagaaa

SEQ ID NO: 33 - DNA, 69 bp, Homo sapiens
tgacgaaccatgcgggattcctcaccatcggggctgatctccactttgccaaagggctgcgagaggtga

SEQ ID NO: 34 - DNA, 69 bp, Homo sapiens
tatcaaggactttcactgcaggcagtgctatttcttgtgcctaagaatgtttccaaaagtcgcatcgct

SEQ ID NO: 35 - DNA, 20 bp, Artificial
  primer
caggaataggagctcggtga

SEQ ID NO: 36 - DNA, 19 bp, Artificial
  primer
ctgggtgctgtgctaggtg

SEQ ID NO: 37 - DNA, 21 bp, Artificial
  primer
tctcttggccaggatcaaaca

SEQ ID NO: 38 - DNA, 20 bp, Artificial
  primer
cagagcctgccatgtactca

SEQ ID NO: 39 - DNA, 20 bp, Artificial
  primer
aaaccaatcatgggaagctg

SEQ ID NO: 40 - DNA, 20 bp, Artificial
  primer
acccgtccttcatcaaactg

SEQ ID NO: 41 - DNA, 20 bp, Artificial
  primer
catcgaccacgagattgaaa

SEQ ID NO: 42 - DNA, 20 bp, Artificial
  primer
ccgctttttcttcaggtgac

SEQ ID NO: 43 - DNA, 20 bp, Artificial
  primer
gagaacagcaagtgcaccaa

SEQ ID NO: 44 - DNA, 20 bp, Artificial
  primer
ttggaatctggagatggagg

SEQ ID NO: 45 - DNA, 20 bp, Artificial
  primer
aaaggctggcacgtttagaa

SEQ ID NO: 46 - DNA, 20 bp, Artificial
  primer
agggaaatcccatcttggtt

SEQ ID NO: 47 - DNA, 20 bp, Artificial
  primer
ccggaaagaagtcagagacg

SEQ ID NO: 48 - DNA, 20 bp, Artificial
  primer
ttgcttctagccgtccattt

SEQ ID NO: 49 - DNA, 20 bp, Artificial
  primer
gaaagaagtcagagacgccg

SEQ ID NO: 50 - DNA, 20 bp, Artificial
  primer
ttgcttctagccgtccattt

SEQ ID NO: 51 - DNA, 20 bp, Artificial
  primer
catgaaggatcctgtggctt

SEQ ID NO: 52 - DNA, 20 bp, Artificial
  primer
caggacaatgctcccaaagt

SEQ ID NO: 53 - DNA, 20 bp, Artificial
  primer
agtgtccaatgtctcctgcc

SEQ ID NO: 54 - DNA, 20 bp, Artificial
  primer
caacaagctcgtccacagaa

(19) United States
(12) Patent Application Publication
(10) Pub. No.: US 2011/0256090 A1
(43) Pub. Date: Oct. 20, 2011

(54) Method for Identifying Genes Involved in Trail-Induced Apoptosis and Therapeutic Applications Thereof

(75) Inventors: Michael Hahne, Montpellier (FR); Bernard Combe, Montpellier (FR); Jacques Morel, Prades Le Lez (FR); Rachel Audo, Montpellier (FR); Alica Knapik, Zug (CH)

(73) Assignees: UNIVERSITE DE MONTPELLIER 2 SCIENCES ET TECHNIQUES, Montpellier Cedex 5 (FR); Centre National de la Pecherche Scientifique- CNRS, Paris Cedex (FR); UNIVERSITE DE MONTPELLIER 1, Montpellier Cedex 2 (FR)

(21) Appl. No.: 13/140,566

(22) PCT Filed: Dec. 18, 2009
(86) PCT No.: PCT/EP2009/067517

§ 371 Date: Jun. 17, 2011

Related U.S. Application Data
(60) Provisional application No. 61/138,785, filed on Dec. 18, 2008.

(51) Int. Cl. A61K 38/19; C12N 5/071; C12N 15/63; C07H 21/02; A61P 35/00; A61K 38/17; A61K 31/713; A61K 31/7088; A61P 29/00; C40B 30/04; C12Q 1/68; C12N 15/85

(52) U.S. Cl. 424/85.1; 435/6.1; 435/375; 435/320.1; 536/24.5; 435/455; 514/18.9; 514/44 A; 514/44 R; 514/16.6; 506/9

(57) ABSTRACT

The invention relates to methods for identifying genes involved in TRAIL-induced apoptosis, to inhibitors of the expression of genes inducing resistance of cells to TRAIL-induced apoptosis and to activators of the expression of a gene sensitizing cells to TRAIL-induced apoptosis. The invention also relates to methods for sensitizing cells to TRAIL-induced apoptosis, methods for treating hyperproliferative diseases, methods for determining the responsiveness of a subject suffering from a hyperproliferative disease to TRAIL, to pharmaceutical compositions comprising products capable of sensitizing cells to TRAIL-induced apoptosis, and to methods for determining the prognosis of a subject suffering from a hyperproliferative disease.

### FIELD OF THE INVENTION

The present invention relates to a method for identifying genes involved in TRAIL-induced apoptosis, and therapeutic applications thereof.

### BACKGROUND OF THE INVENTION

In recent years, considerable attention has been focused on the potential benefits of TRAIL (TNF-related apoptosis inducing ligand) in cancer therapy, as a broad range of cancer cells are sensitive to TRAIL-induced apoptosis (Wang, S et al. (2003) Oncogene 22: 8628-33). In addition, the use of TRAIL in combination with chemotherapeutic agents or irradiation strengthens its apoptotic effects and frequently sensitizes otherwise TRAIL-resistant cancer cells. Importantly, TRAIL does not appear to be toxic to normal cells, as TRAIL-exposure shows no toxic side effects of therapeutically relevant doses in primates.

TRAIL can interact with five different receptors: four membrane-anchored receptors TRAIL-R1 (DR4), TRAIL-R2 (DR5), TRAIL-R3 (DcR1) and TRAIL-R4 (DcR2) and a soluble decoy receptor osteoprotegerin (OPG). The receptors TRAIL-R1 and -R2 contain an intracellular cytoplasmic sequence motif, known as the death domain (DD), and can induce apoptosis through activation of caspases (Di Pietro et al. (2004) J Cell Physiol 201: 331-40). Nevertheless, TRAIL-receptors R1 and R2 not only trigger apoptosis, but also proliferation and differentiation depending on the cell type (Di Pietro et al., 2004). This phenomenon has been described for several other members of the TNF family and it is thought that one pathway potentially pre-dominates but that a buildup of intracellular regulators can flick the switch from cell death to proliferation and viceversa (Di Pietro et al., 2004; Screaton et al. (2000) Curr Opin Immunol 12: 316-22). For example, TRAIL has been shown to promote cell survival and proliferation of endothelial and vascular smooth muscle cells (Secchiero, P et al. (2003) Circulation 107: 2250-6; Secchiero, P et al. (2004) Cell Mol Life Sci 61: 1965-74) and to regulate erythroid and monocytic maturation (Secchiero, P et al. (2004) Blood 103: 517-22).

The role of TRAIL has been also studied in Rheumatoid arthritis. Rheumatoid arthritis (RA) (Pope, R. M. (2002) Nat. Rev. Immunol. 2, 527-535) is an autoimmune disease characterized by chronic inflammation of joints leading to progressive and irreversible joint destruction. The aggressive front of synovial tissue, called pannus, invades and destroys local articular structure. The pannus is characterized by a synovial hyperplasia that is mainly composed of fibroblast-like synoviocytes (FLSs) combined with a massive infiltration of lymphocytes and macrophages. Both increased proliferation and/or insufficient apoptosis might contribute to the expansion of RA FLSs, and several reports suggest inducing apoptosis of RA FLSs as a therapeutic approach. It has been described that TRAIL induces apoptosis only in a subset of RA FLS that is followed by an induction of proliferation in the surviving cells (Morel et al. (2005), J. Biol. Chem. 280: 15709-15718). This suggests that FLS of RA patients consists of different subpopulations according to their different TRAIL-responses.

Evidence is accumulating that TRAIL has multiple effects also on cancer cells. For example, Erhardt et al. analyzed the effect of TRAIL on primary cells of children with untreated acute leukemia (Ehrhardt, H et al. (2003) Oncogene 22: 3842-52). They observed that TRAIL induced apoptosis only in 50% of the leukemia cell samples tested, but survival or proliferation on the remaining samples (Ehrhardt, H et al., 2003). Concurring with this report is a study describing that the effect of TRAIL on leukemia cells can be either pro-apoptotic or pro-proliferative (Baader et al. (2005) Cancer Res 65: 7888-95). A more recent publication reported that TRAIL promotes metastasis of human pancreatic ductal adenocarcinoma in SCID/beige mice (Trauzold, A et al. (2006) TRAIL promotes metastasis of human pancreatic ductal adenocarcinoma, Oncogene).

All these findings challenge the proposed strategy to use TRAIL for targeting hyperproliferative cells and there is thus a need of new strategies alternative or complementary to the TRAIL strategy used to date.

### SUMMARY OF THE INVENTION

The invention first relates to methods for identifying genes involved in TRAIL-induced apoptosis in a population of cells comprising the steps of:
1) contacting said population of cells with TRAIL,
2) isolating the subset of cells of the population which are sensitive to TRAIL-induced apoptosis (sensitive subset) and the subset of cells of the population which are resistant to TRAIL-induced apoptosis (resistant subset),
3) comparing the gene expression in the sensitive subset and in the resistant subset, and
4) identifying the genes that are differentially expressed in the sensitive subset and in the resistant subset, the genes being over expressed in the sensitive subset being classified as genes sensitizing the cells of said population to TRAIL-induced apoptosis and the genes being over expressed in the resistant subset being classified as genes inducing resistance of the cells of said population to TRAIL-induced apoptosis.

The invention also relates to inhibitors of the expression of a gene inducing resistance of cells to TRAIL-induced apoptosis, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5.

The invention still relates to activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ D NO:13, SEQ ID NO:14, SEQ ID NO:15 and SEQ ID NO:16.

The invention also relates to isolated nucleotide sequences selected from the group comprising SEQ ID NO:17, SEQ ID NO:18, SEQ ID NO:19, SEQ ID NO:20, SEQ ID NO:21 and SEQ ID NO:22.

The invention also relates to in vitro methods for sensitizing cells to TRAIL-induced apoptosis, said method comprising the step of contacting said cells with a product capable of sensitizing cells to TRAIL-induced apoptosis, wherein said product is selected from the group comprising:
inhibitors of the expression of a gene inducing resistance of cells to TRAIL according to the invention,
activators of the expression of a gene sensitizing cells to TRAIL-induced apoptosis according to the invention,
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

The invention still relates to products capable of sensitizing cells to TRAIL-induced apoptosis for use in a method for sensitizing cells to TRAIL-induced apoptosis in a human or animal body, wherein said product is selected from the group comprising:
inhibitors of the expression of a gene inducing resistance of cells to TRAIL according to the invention,
activators of the expression of a gene sensitizing cells to TRAIL-induced apoptosis according to the invention,
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO: 16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ED NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

The invention further relates to products capable of sensitizing cells to TRAIL-induced apoptosis for use in a method for treating a hyperproliferative disease in a human or animal body, wherein said product is selected from the group comprising:
inhibitors of the expression of a gene inducing resistance of cells to TRAIL according to the invention,
activators of the expression of a gene sensitizing cells to TRAIL-induced apoptosis according to the invention,
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

The invention still relates to methods for determining the responsiveness of a subject suffering from a hyperproliferative disease to TRAIL, comprising the step of detecting, in hyperproliferative cells obtained from said subject, the expression of a gene inducing resistance of said cells to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, and wherein the detection of the expression of a gene inducing resistance of said cells to TRAIL-induced apoptosis is indicative of poor response of said subject to TRAIL.

The invention also relates to methods for determining the responsiveness of a subject suffering from a hyperproliferative disease to TRAIL, comprising the step of detecting, in hyperproliferative cells obtained from said subject, the expression of a gene sensitizing said cells to TRAIL-induced apoptosis wherein said gene comprises a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and wherein the detection of the expression of a gene sensitizing said cells to TRAIL-induced apoptosis is indicative of good response of said subject to TRAIL.

The invention still relates to pharmaceutical compositions comprising a product capable of sensitizing cells to TRAIL-induced apoptosis, together with a pharmaceutically acceptable carrier, wherein said product is selected from the group comprising:
inhibitors of the expression of a gene inducing resistance of cells to TRAIL as defined in claim 2 or 3,
activators of the expression of a gene sensitizing cells to TRAIL-induced apoptosis as defined in claim 4,
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

The invention further relates to methods for determining the prognosis of a subject suffering from a hyperproliferative disease, comprising the step of detecting, in a sample obtained from said subject, the expression of a gene inducing resistance to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, wherein said expression indicates that the subject has a poor prognosis.

The invention also relates to methods for determining the prognosis of a subject suffering from a hyperproliferative disease, comprising the step of detecting, in a sample obtained from said subject, the expression of a gene sensitizing said cells to TRAIL-induced apoptosis wherein said gene comprises a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, wherein said expression indicates that the subject has a good prognosis.

### DEFINITIONS

Applicant intends to utilize the definitions of the terms and expressions provided herein, unless specifically indicated otherwise.

### DETAILED DESCRIPTION OF THE INVENTION

In a first aspect, the invention relates to a method for identifying genes involved in TRAIL-induced apoptosis in a population of cells comprising the steps of:
1) contacting said population of cells with TRAIL,
2) isolating the subset of cells of the population which are sensitive to TRAIL-induced apoptosis (sensitive subset) and the subset of cells of the population which are resistant to TRAIL-induced apoptosis (resistant subset),
3) comparing the gene expression in the sensitive subset and in the resistant subset, and
4) identifying the genes that are differentially expressed in the sensitive subset and in the resistant subset, the genes being over expressed in the sensitive subset being classified as genes sensitizing the cells of said population to TRAIL-induced apoptosis and the genes being over expressed in the resistant subset being classified as genes inducing resistance of the cells of said population to TRAIL-induced apoptosis.

As used herein, “population of cells” means any type of cells susceptible to be the target of a TRAIL treatment strategy, in particular hyperproliferative cells. Non limitative examples of populations of cells according to the invention are cancer cells and rheumatoid arthritis fibroblast-like synoviocytes (RA-FLS).

According to the invention, step (1) of the method hereinabove described is performed by incubating said population of cells with TRAIL by any suitable method known by the skilled person. For instance, the cells may be incubated in 12-well plates, each well comprising about 1·10⁵ cells, during 12-24 hours, which corresponds to the average time for obtaining maximal apoptosis. The concentration of TRAIL which can be used for incubating the cells is typically in the range from 0.1 nM to 10 nM, particularly about 1 nM.

According to the invention, step (2) of the method hereinabove described is performed by any apoptosis detection method known by the skilled person. These methods are numerous, fully described in the art, kits thereof are commercially available, and the skilled person is thus able to select the most appropriate method. Examples of methods for detecting apoptosis in a cell are methods based on the natural property of annexin V to interact with phosphatidylserine (PS): most of the phosphatidylserines (PS) in cell membrane phospholipids translocate from the inner surface to the outer surface during the early stages of apoptosis. Once the PS are on the outer surface, they can be detected easily by staining with a fluorescent protein fused with annexin V, e.g. by Fluorescence-activated cell sorting (FACS). Annexin V can also be labelled with colloid gold for electron microscopy, with radioactive tracer for autoradiography on the tissue level and with peroxidase for histochemical studies. Obviously, other methods can be used to detect apoptosis in a cell, such as for example the detection of activated caspases, e.g. with caspase inhibitors conjugated to a fluorescence marker, or the detection of change in mitochondrial transmembrane potential, e.g. by FACS or fluorescence microscopy.

According to the invention, step (3) of the method hereinabove described is performed by any known gene expression profiling method. A gene expression profiling method consists in the measurement of the expression of thousands of genes at once, to create a global picture of cellular function. These profiles can, for example, distinguish between cells that are actively dividing, or show how the cells react to a particular treatment. Many methods of this sort measure an entire genome simultaneously, that is, every gene present in a particular cell. The most common and well known method that can be used according to the invention for gene expression profiling is DNA microarray. Microarrays are commercially available and the skilled person is able to select the most appropriate microarray to the study of a particular population of cells. Tag-based techniques, like serial analysis of gene expression (SAGE, SuperSAGE, see Velculescu V E et al. (1995) Science 270 (5235): 484-7; Saha S et al. (2002) Nat Biotechnol 20 (5): 508-12; Gowda M. et al. (2004) Plant Physiol 134 (3): 890-7; Matsumura H. et al. (2005). Cell Microbiol 7 (1): 11-8) may also be used for gene expression profiling. Another method is deep sequencing, which is an emerging alternative to microarray gene profiling (Burnside J. et al (April 2008) BMC Genomics 9 (1): 185).

According to the invention, the differential expression of the genes is typically measured with a linear model for microarray data package, or LIMMA package (Bioconductor). LIMMA is a software package for the analysis of gene expression microarray data, especially the use of linear models for analysing designed experiments and the assessment of differential expression. The package includes pre-processing capabilities for two-colour spotted arrays. The differential expression methods apply to all array platforms and treat Affymetrix, single channel and two channel experiments in a unified way. (Gentleman R C et al. Genome Biol 2004, 5: R80; http://www.bioconductor.org/; Smyth, G. K. et al. (2003) Methods 31, 265-273; Smyth, G. K. (2004) Statistical Applications in Genetics and Molecular Biology 3, No. 1, Article 3; Smyth, G. K. (2005) in: Bioinformatics and Computational Biology Solutions using R and Bioconductor, R. Gentleman, et al., Springer, N.Y., pages 397-420; R. Gentleman, V. et al. Springer, N.Y., pages 397-420; http://bioinfwehi.edu.au/limma/; Tusker V. G. et al., PNAS 2001 Apr. 24; 98(9):5116-21).

In a particular embodiment, a gene is considered as “differentially expressed” between two subsets of cells when the probability of having a differential expression between said subsets is greater than 60%, as measured by the statistical method as defined above.

In one embodiment of the invention, results obtained by the gene expression profiling as described previously are validated by QPCR (Quantitative real time polymerase chain reaction) or RTPCR (Reverse Transcription PCR), as classically described in the art. Other experiments, such as a western blot of some of the protein products of differentially expressed genes, can also be performed to confirm the conclusions based on the expression profile.

In a particular embodiment, the method for identifying genes hereinabove described is directed to cancer cells. In this particular embodiment, the method for identifying genes involved in TRAIL-induced apoptosis in cancer cells comprises the particular steps of:
1) contacting said cancer cells with TRAIL,
2) isolating the cancer cells which are sensitive to TRAIL-induced apoptosis (sensitive cells) and the cancer cells which are resistant to TRAIL-induced apoptosis (resistant cells),
3) comparing the gene expression in the sensitive cells and in the resistant cells, and
4) identifying the genes that are differentially expressed in the sensitive cells and in the resistant cells, the genes being over expressed in the sensitive cells being classified as genes sensitizing the cancer cells to TRAIL-induced apoptosis and the genes being over expressed in the resistant cells being classified as genes inducing resistance of the cancer cells to TRAIL-induced apoptosis.

In another embodiment, the method for identifying genes hereinabove described is directed to Rheumatoid Arthritis Fibroblast-Like Synoviocytes (RA-FLS). In this particular embodiment, the method for identifying genes involved in TRAIL-induced apoptosis in RA-FLS comprises the particular steps of:
1) contacting RA-FLS with TRAIL,
2) isolating the RA-FLS which are sensitive to TRAIL-induced apoptosis (RA-FLS-S) and the RA-FLS which are resistant to TRAIL-induced apoptosis (RA-FLS-R),
3) comparing the gene expression in the RA-FLS-S and in the RA-FLS-R, and
4) identifying the genes that are differentially expressed in the RA-FLS-S and in the RA-FLS-R, the genes being over expressed in the RA-FLS-S being classified as genes sensitizing RA-FLS to TRAIL-induced apoptosis and the genes being over expressed in RA-FLS-R being classified as genes inducing resistance of RA-FLS to TRAIL-induced apoptosis.

Examples of genes inducing resistance of the cells to TRAIL-induced apoptosis identified by the method according to the invention comprise the nucleotide sequence as shown in SEQ ID NO:1, SEQ ID NO:2, SEQ ID NO:3, SEQ ID NO:4 or SEQ ID NO:5.

In a particular embodiment, the genes inducing resistance of the cells to TRAIL-induced apoptosis typically comprise a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5.

Examples of genes sensitizing the cells to TRAIL-induced apoptosis identified by the method according to the invention comprise a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

Typically, genes sensitizing the cells to TRAIL-induced apoptosis typically comprise a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

According to the invention, to determine the percent identity of two nucleic acid sequences, the sequences are aligned for optimal comparison. For example, gaps can be introduced in the sequence of a first nucleic acid sequence for optimal alignment with the second nucleic acid sequence. The nucleotides at corresponding nucleotide positions are then compared. When a position in the first sequence is occupied by the same nucleotide as at the corresponding position in the second sequence, the nucleic acids are identical at that position. The percent identity between the two sequences is a function of the number of identical nucleotides shared by the sequences.

Hence % identity=[number of identical nucleotides/total number of overlapping positions]×100. The percentage of sequence identity is thus calculated according to this formula, by comparing two optimally aligned sequences over the window of comparison, determining the number of positions at which the identical nucleic acid base (e. g., A, T, C, G) occurs in both sequences to yield the number of matched positions (the “number of identical positions” in the formula above), dividing the number of matched positions by the total number of positions in the window of comparison (e.g. the window size) (the “total number of overlapping positions” in the formula above), and multiplying the result by 100 to yield the percentage of sequence identity.

In this comparison, the sequences can be the same length or may be different in length. Optimal alignment of sequences for determining a comparison window may be conducted by the local homology algorithm of Smith and Waterman (1981), by the homology alignment algorithm of Needleman and Wunsh (1972), by the search for similarity via the method of Pearson and Lipman (1988), by computerized implementations of these algorithms (GAP, BESTFIT, FASTA and TFASTA in the Wisconsin Genetics Software Package Release 7.0, Genetic Computer Group, 575, Science Drive, Madison, Wis.), or by inspection.

The invention also relates to inhibitors of the expression of a gene inducing resistance of cells to TRAIL-induced apoptosis, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5.

According to the invention, an inhibitor of the expression of a gene inducing resistance of cells to TRAIL-induced apoptosis is typically a nucleic acid which interferes with the expression of said gene. Examples of such inhibitors are antisense molecules or vectors comprising said antisense molecules. Antisense molecules are complementary strands of small segments of mRNA. Methods for designing effective antisense molecules being well known (see for example U.S. Pat. No. 6,165,990), it falls within the ability of the skilled artisan to design antisense molecules able to downregulate the expression of a gene inducing resistance of the hereinabove defined cells to TRAIL-induced apoptosis. Further examples are RNA interference (RNAi) molecules such as, for example, short interfering RNAs (siRNAs) and short hairpin RNAs (shRNAs). siRNA refers to the introduction of homologous double stranded RNA to specifically target a gene's product, in the present case a gene inducing resistance of cells to TRAIL-induced apoptosis, resulting in a null or hypomorphic phenotype. Methods for designing effective RNAi molecules being well known (see for review Hannon and Rossi Nature. 2004 Sep. 16; 431(7006):371-8), it falls within the ability of the skilled artisan to design RNAi molecules able to downregulate the expression of IL4I1 in IL4I1-expressing cells.

In a particular embodiment of the invention, the inhibitor of the expression of a gene inducing resistance of cells to TRAIL-induced apoptosis is a siRNA comprising a nucleotide sequence selected from the group comprising SEQ ID NO:17, SEQ ID NO:18, SEQ ID NO:19, SEQ ID NO:20, SEQ ID NO:21 and SEQ ID NO:22.

The invention also relates to isolated nucleotide sequences selected from the group comprising SEQ ID NO:17, SEQ ID NO:18, SEQ ID NO:19, SEQ ID NO:20, SEQ ID NO:21 and SEQ ID NO:22.

The invention still relates to activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

According to the invention, an activator of the expression of a gene inducing resistance of cells to TRAIL-induced apoptosis are typically activators of mitogen-activated protein kinases (MAPK), PI3-kinases or cytokines such as IL-8.

The invention also relates to expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ED NO:11, SEQ ED NO:12, SEQ ID NO:13, SEQ ID NO:14, SEQ ID NO:15 and SEQ ID NO:16.

As used herein, the terms “expression vector” refer to a nucleic acid molecule capable of directing the expression of a given nucleic acid sequence which is operatively linked to an expression control sequence or promoter. In particular, an expression vector according to the invention is a vector which enables the expression of a given nucleic acid sequence into the protein encoded by said nucleic acid sequence in a eukaryotic host cell. The promoter of said expression vector is typically a eukaryotic promoter. An expression vector according to the invention enables the expression of a protein able to sensitize cells to TRAIL-induced apoptosis.

The expression vector(s) of the present invention can be a plasmid or a viral vector. A plasmid is a circular double-stranded DNA loop that is capable of autonomous replication. A viral vector is a nucleic acid molecule which comprises viral sequences which can be packaged into viral particles. A variety of viral vectors are known in the art and may be adapted to the practice of this invention, including e.g., adenovirus, AAV, retrovirus, hybrid adeno-AAV, lentivirus and others. By carrying out routine experimentation, the skilled person in the art can chose from the variety of available vectors, those which are suitable for carrying out the method of the invention.

The invention further relates to proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

The invention also relates to methods for sensitizing to TRAIL-induced apoptosis cells which are resistant to TRAIL-induced apoptosis.

The inventions thus relates to in vitro methods for sensitizing cells to TRAIL-induced apoptosis, said method comprising the step of contacting said cells with a product capable of sensitizing cells to TRAIL-induced apoptosis.

According to the invention, a “product capable of sensitizing cells to TRAIL-induced apoptosis” is a product selected from the group comprising an inhibitor of the expression of a gene inducing resistance of cells to TRAIL according to the invention, an activator of the expression of a gene sensitizing cells to TRAIL-induced apoptosis according to the invention, an expression vector according to the invention, and a protein able to sensitize cells to TRAIL-induced apoptosis according to the invention.

The invention still relates to products capable of sensitizing cells to TRAIL-induced apoptosis according to the invention, for use in a method for sensitizing cells to TRAIL-induced apoptosis in a human or animal body.

In a particular embodiment, the cells resistant to TRAIL-induced apoptosis are cancer cells. In this particular embodiment the invention thus pertains to methods for sensitizing cancer cells to TRAIL-induced apoptosis.

In another particular embodiment, the cells resistant to TRAIL-induced apoptosis are Rheumatoid Arthritis Fibroblast-Like Synoviocytes (RA-FLS). In this particular embodiment, the invention thus pertains to methods for sensitizing RA-FLS to TRAIL-induced apoptosis.

In still another aspect, the invention relates to methods for treating a hyperproliferative disease comprising administering to a subject in need thereof an effective amount of a product capable of sensitizing cells to TRAIL-induced apoptosis according to the invention.

The invention also relates to products capable of sensitizing cells to TRAIL-induced apoptosis according to the invention, for use in a method for treating a hyperproliferative disease in a human or animal body.

As used herein, “hyperproliferative disease” means a disease resulting from rapid cell division. Hyperproliferative diseases include, but are not limited to, cancer, rheumatoid arthritis, psoriasis, actinic keratosis and lamellar ichthyosis, systemic lupus erythematosus (SLE).

In a particular embodiment of the invention, the hyperproliferative disease to be treated is cancer. In this embodiment, the cells to be treated are cancer cells. As used herein, “cancer” means all types of cancers. In particular, the cancers can be solid or non solid cancers. Non limitative examples of cancers are carcinomas such as breast, prostate, lung or colon cancer, sarcomas, lymphomas, leukemias, germ cell cancers and blastomas.

In another particular embodiment, the hyperproliferative to be treated is rheumatoid arthritis. In this embodiment, the cells to be treated are FLS.

In one embodiment, the methods for treating a hyperproliferative disease according to the invention further comprise the simultaneous, sequential or separate administration of an effective amount of TRAIL in said subject.

In another embodiment, the methods for treating cancer according to the invention, are applied to the human or animal body simultaneously, separately or sequentially with another method for treating cancer. Said another method for treating cancer is typically selected from the group comprising surgery, external radiotherapy, chemotherapy, hormone therapy and cytokine therapy. In a particular embodiment, the method for treating cancer according to the invention is combined with a chemotherapy, wherein said chemotherapy comprises the administration of at least one anti-cancer agent.

As used herein, the expression “anti-cancer agent” refers to compounds which are used in the treatment of cancer. In particular, the expression “anti-cancer, agent” refers to compounds that were reported to synergise with TRAIL-induced apoptosis. These reagents include DNA modulators (such as cisplatin), histone deacetylase inhibitors, P13 kinase pathway inhibitors, NFkappaB inhibitors, IAP (inhibitor of apoptosis protein) (Johnstone, R. W. et al. 2008, Nat Rev Cancer 8:782-798). Particular anti-cancer agents according to the invention include but are not limited to fludarabine, gemcitabine, capecitabine, methotrexate, taxol, taxotere, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbizinc, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnclamine, topotecan, kinase inhibitors, phosphatase inhibitors, ATPase inhibitors, tyrphostins, protease inhibitors, inhibitors herbimycm A, genistein, erbstatin, and lavendustin.

In one embodiment, the anti-cancer agent is selected for the group consisting of taxol; taxotere; platinum complexes such as cisplatin, carboplatin and oxaliplatin; doxorubicin; taxanes such as docetaxel and paclitaxel; vinca alkaloids such as vinblastine, vincristine and vinorelbine; genistein; erbstatin; and lavendustin.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of cancer.

As used herein, “subject” refers to a human or animal that may benefit from the administration of a compound, a composition or a method as recited herein. Most often, the subject will be a human but can be any mammals.

By “compound” it is meant an inhibitor of the expression of a gene inducing resistance of hyperproliferative cells to TRAIL-induced apoptosis identified by the method as defined hereinabove or an activator of the expression of a gene sensitizing hyperproliferative cells to TRAIL-induced apoptosis identified by the method as defined hereinabove.

By a “therapeutically effective amount” of a compound as described previously, is meant a sufficient amount to treat a disease, at a reasonable benefit/risk ratio applicable to any medical treatment. It will be understood, however, that the total daily usage of a compound according to the invention will be decided by the attending physician within the scope of sound medical judgment. The specific therapeutically effective dose level for any particular subject in need thereof will depend upon a variety of factors including the stage of the disease being treated, the age, body weight, general health, sex and diet of the subject, the time of administration, route of administration, the duration of the treatment; drugs used in combination or coincidental with the and like factors well known in the medical arts. For example, it is well known within the skill of the art to start doses of a compound at levels lower than those required to achieve the desired therapeutic effect and to gradually increase the dosage until the desired effect is achieved.

The invention still relates to methods for determining the responsiveness of a subject suffering from a hyperproliferative disease to TRAIL, comprising the step of detecting, in hyperproliferative cells obtained from said subject, the expression of a gene inducing resistance of said cells to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, and wherein the detection of the expression of a gene inducing resistance of said cells to TRAIL-induced apoptosis indicates that said subject is responsive to TRAIL.

The invention also relates to methods for determining the responsiveness of a subject suffering from a hyperproliferative disease to TRAIL, comprising the step of detecting, in hyperproliferative cells obtained from said subject, the expression of a gene sensitizing said cells to TRAIL-induced apoptosis wherein said gene comprises a nucleotide sequence as shown in SEQ NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and wherein the detection of the expression of a gene sensitizing said cells to TRAIL-induced apoptosis indicates that said subject is not responsive to TRAIL.

In a particular embodiment, the hyperproliferative disease is cancer. Examples of samples obtained from the subjects are any type of cancer biopsy, including lymph nodes, and optionally whole blood sample.

In another particular embodiment, the hyperproliferative is rheumatoid arthritis. Examples of samples obtained from a subject suffering from rheumatoid arthritis are typically biopsies of synovial tissue or synovial liquid.

In the methods for determining the responsiveness of a subject suffering from a hyperproliferative disease to TRAIL according to the invention, a subject will be considered to be responsive, i.e. sensitive, to TRAIL if the expression of a gene sensitizing the cells to TRAIL-induced apoptosis is detected. To the contrary, a subject will be considered to be non responsive, i.e. resistant, to TRAIL if the expression of a gene inducing resistance of the cells to TRAIL-induced apoptosis is detected.

It falls within the ability of the skilled person to carry out the detection of the expression of a gene according to the invention. Indeed, such expression can be detected by any method known by the skilled person. In particular, the expression may be determined using RT-PCR and QPCR. The expression may also be detected by immunological techniques such as ELISA and Western Blot, for example on biological fluids (whole blood sample, plasma sample, serum sample, synovial liquid sample etc. . . . ).

The invention still relates to pharmaceutical compositions comprising a product capable of sensitizing cells to TRAIL-induced apoptosis according to the invention, together with a pharmaceutically acceptable carrier.

By “comprising a product” it is meant that the composition can comprise one or several products capable of sensitizing cells to TRAIL-induced apoptosis according to the invention.

In a particular embodiment, the pharmaceutical composition according to the invention further comprises TRAIL.

In another aspect, the invention relates to the composition according to the invention for use in a method for treating a hyperproliferative disease.

In another aspect, the invention pertains to a product comprising
TRAIL, and
a product capable of sensitizing cells to TRAIL-induced apoptosis according to the invention,
   as a combined preparation for simultaneous, separate or sequential use in a method for treating a hyperproliferative disease in the human or animal body.

In one embodiment, the hyperproliferative cells according to the invention are selected from the group comprising cancer cells and rheumatoid arthritis fibroblast-like synoviocytes (RA-FLS).

The invention also relates to methods for determining the prognosis of a subject suffering from a hyperproliferative disease, comprising the step of detecting, in a sample obtained from said subject, the expression of a gene inducing resistance to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, wherein said expression indicates that the subject has a poor prognosis.

The invention still relates to methods for determining the prognosis of a subject suffering from a hyperproliferative disease, comprising the step of detecting, in a sample obtained from said subject, the expression of a gene sensitizing said cells to TRAIL-induced apoptosis wherein said gene comprises a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, wherein said expression indicates that the subject has a good prognosis.

In a particular embodiment, said hyperproliferative disease is cancer. In this embodiment, examples of samples obtained from the subjects are any type of cancer biopsy, including lymph nodes, and optionally whole blood sample.

In another particular embodiment, the hyperproliferative is rheumatoid arthritis. In this embodiment, examples of samples obtained from a subject suffering from rheumatoid arthritis are typically biopsies of synovial tissue or synovial liquid.

In the methods for determining the prognosis according to the invention, the detection of the expression of said genes can be carried out by detecting the presence of mRNAs of said genes in the cells of the samples, notably by RT-PCR, or any other method known by the skilled person, such as QPCR and immunological techniques such as ELISA and Western Blot, for example on biological fluids (whole blood sample, plasma sample, serum sample, synovial liquid sample etc. . . . ).

The term “detecting” as used in the invention includes qualitative and/or quantitative detection (measuring levels) with or without reference to a control.

The term “prognosis” is used herein to refer to the prediction of the likelihood of death or progression attributable to the hyperproliferative disease. Progression includes recurrence, metastatic spread, and drug resistance.

As used herein, “poor prognosis” indicates an increased likelihood of death or progression attributable to the hyperproliferative disease.

As used herein, “good prognosis” indicates a decreased likelihood of death or progression attributable to the hyperproliferative disease.

The prognosis results obtained according to the method of the invention can also be correlated to, or serve as a basis for, a “risk classification” of the patients. As used herein, “risk classification” means the level of risk or the prediction that a subject will experience a particular clinical outcome. A subject may be classified into a risk group or classified at a level of risk based on the predictive methods of the present invention. A “risk group” is a group of subjects or individuals with a similar level of risk for a particular clinical outcome.

The present invention is better illustrated below using the examples which follow. These examples are given only by way of illustration of the subject-matter of the invention, of which they in no way constitute a limitation.

### FIGURES

FIG. 1: “DICER SUITE” Diagram. The mRNA of the FLS-S and FLS-R are hybridised two by two on a single plate. Each FLS-S will thus be hybridised with 2 FLS-R and vice-versa, with a final total of 12 hybridisations.

FIG. 2: A. Response of the FLS isolated from synovial tissues of women (1: apoptosis, −1: no/little apoptosis) in function of their ages. Y axis: Response to TRAIL; X axis: Age of the patients. B. Susceptibility of primary cultures of FLS to TRAIL-induced apoptosis (Y axis) correlates with disease activity of rheumatoid arthritis patients (DAS28; X axis). TRAIL-induced apoptosis on FLS was determined by FACS analysis as described below.

FIG. 3: comparison of the expression of GALNT1, SULF2, Acheron and Liprin by quantitative PCR. The mean expression in each group (FLS-R and FLS-S) is compared to the average of the totality of patients (controls noted here). *p<0.05, Wilcoxon test, n=6.

FIG. 4: Analysis of the effect of siRNA targeting the expression of GALNT-1 and SULF-2 on TRAIL-induced apotosis. The cells are transfected with the siRNA which target GALNT-1, SULF-2 or a control siRNA for 60 h then stimulated with TRAIL for 24 h. The % of apotosis is measured by FACS by means of the annexin V fixation test and incorporation of TOPRO-3. The results are expressed in % of total cell death (*p<0.05, Wilcoxon test, n=5). The box shows a reduction in the coding mRNA for GALNT1 and SULF in the FLS treated with the siRNA which target them compared to the cells treated with the control siRNA.

FIG. 5: Analysis of the effect of siRNA targeting the expression of ORP-4 on TRAIL-induced apoptosis. The cells are transfected with the siRNA which target ORP-4, a control siRNA or only the transfection reagent for 60 hours then stimulated with TRAIL for 24 hours. The % of apoptosis is measured by FACS by means of the annexin V fixation test and incorporation of TOPRO-3. The results are expressed as a % of total cell death (**p<0.01, Wilcoxon test, n=9).

FIG. 6: Comparison of expression levels of SEQ ID N^{o} 15 (PLTP) all isoforms (A), SEQ ID N^{o} 15 (PLTP) isoform 1 (B), SEQ ID N^{o} 4 (EIF1AX) isoform 1 and 2 (C), SEQ ID N^{o} 4 isoform 2 (D), SEQ ID N^{o} 9 (SULF2) (E) and SEQ ID NO 3 (liprin-β1) (F) by quantitative PCR between FLS-S (S) and FLS-R (R). mRNA levels were expressed in Arbitrary Units (AU) vs β-2 microglobulin expression. The mean in each group is compared between FLS-R and FLS-S using the Mann-Whitney test.

FIG. 7: Comparison of PLTP activity in synovial fluid from rheumatoid arthritis (RA) patients and osteoarthritis (OA) patients.

### EXAMPLES

In the following description, all molecular biology experiments for which no detailed protocol is given are performed according to standard protocols.

### Material and Methods

### Biological Material

The fibroblastic cells are isolated from a synovial membrane biopsy of patients with RA (Morel, J. et al. 2005. J Biol Chem 280:15709-15718). The sensitivity to TRAIL-induced apoptosis of the different cultures thus established is evaluated by means of the annexin V test. Depending on the percentage of TRAIL-induced apoptosis, the synoviocytes are classed in 2 groups presenting high (30-50%) or low (0-10%) sensitivity to TRAIL-induced apoptosis.

The total proteins and RNA are extracted after progressive deprivation in serum (5% then 1%) as described previously for the stimulation experiments with TRAIL (Morel, J. et al. 2005. J Biol Chem 280:15709-15718). The sensitivity of the FLS to TRAIL-induced apoptosis is measured in parallel. The sensitive or resistant nature of the FLS is validated in two apoptosis measurement experiments.

### Measurement of Apoptosis

The apoptosis experiments are carried out on a 12 well assay plate, corresponding to around 1×10⁵ cells/well. The cells are treated for 12 and 24 hours, which correspond to the time when the maximum apoptosis is observed. After stimulation, the cells in suspension and adherent are collected, washed twice in cold PBS 2% BSA (to preserve the cells in suspension, well separated and to limit cell death linked to manipulation). The cells are then resuspended in 100 μl of Annexin V-Fluos (Roche). The cells are incubated for 15 min on ice. A volume of 150 μl of ABB buffer containing TOPRO-3 (Molecular Probes) is added, then the cells are analysed in the FASCalibur which measures the fluorescence associated with annexin V-FITC (emission measured at 520 nm) and TOPRO-3 (emission measured at 660 nm). TOPRO-3 is a DNA intercalant which makes it possible to mark the permeable cells and thus to distinguish the necrotic cells and the cells in the final phase of apoptosis.

### Microarrays

Extraction of Messenger RNA (mRNA)

The total RNA are extracted by means of the TRIZol method (Invitrogen, Cergy Pontoise, France)) and purified by precipitation in the LiCl. The purity of the mRNA thus obtained is verified by Agilent Bioanalyser.

The total RNA are then taken over by the transcriptome platform of “Montpellier LR Genopole”. The transcriptome analysis by the DNA chip technique (spotting, hybridations, scans and statistical processing) is carried out by the platform personnel. The chips used are the “Human V4 OpArray” chips containing 35,035 probes representing ˜25,100 genes and 39,600 transcripts.

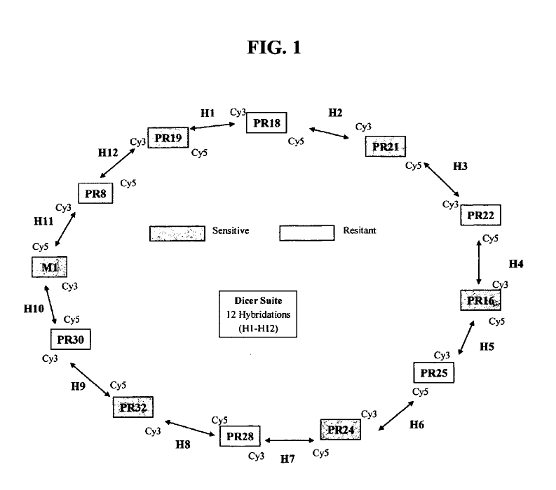

The FLS of different patients are compared on the Dicer suite model (FIG. 1), making it possible to compare the transcriptomes two by two.

### Quantitative PCR

The mRNA are extracted by the TRIZol method, and the reverse transcription reaction is performed by means of the SUPERSCRIPT™ II RNAse H-RT kit (Invitrogen) according to the protocol supplied. The cDNA thus synthesised is then analysed by quantitative PCR.

One of the critical phases of this experiment is the choice of the PCR primers. The selection of the sequences serving as primers is done by means of the “primer 3” software (Rozen and Skaletsky 2000). This software makes it possible to obtain, from the complete cDNA sequence of the gene to be studied, a list of primer pairs liable to enable the amplification of the targeted gene, with the following criteria: size of the amplicon comprised between 75 and 100 bp, percentage of GC of around 40-50% and a fusion temperature of around 60° C. Furthermore, the primer pairs selected must also obey the same rules as for classic PCR, that is, the difference in fusion temperature (TM) between the primers of the same pair must not exceed 5° C. The oligos are chosen so as to amplify only the cDNA and not the genomic DNA which could contaminate the preparations of total RNA and hence of cDNA. The amplified sequence must therefore overlap over two exons. This condition as well as the specificity of the primer pair for the target gene are verified on the site http://www.ncbi.nlm.nih.gov/BLAST/.

The validity of the primer pair is first verified on a cDNA dilution curve obtained from cells to be tested as described above. The dilution curve enables us to obtain the calibration right, from which the efficacy of the primer pair in the quantitative PCR reaction will be deduced and the specificity of the pair is verified by means of the dissociation curve.

We validated the following primer pairs and the optimal elongation temperatures for each of the genes tested (table 1).

The quantitative PCR reaction is carried out by means of a reaction mixture produced at IGMM and described in 2006 (Luftalla and Uze, 2006).

TABLE 1
List of primer pairs used for the Quantitative PCR

| Gene | Forward (F) primer | ID | Reverse (R) primer | ID |
|---|---|---|---|---|
| LARP6 (Acheron#1) | CAGGAATAGGAGCTCGGTGA | 35 | CTGGGTGCTGTGCTAGGTG | 36 |
| GALNT1#1 | TCTCTTGGCCAGGATCAAACA | 37 | CAGAGCCTGCCATGTACTCA | 38 |
| Liprinβ1 isoform1 | AAACCAATCATGGGAAGCTG | 39 | ACCCGTCCTTCATCAAACTG | 40 |
| Liprinβ1 isoform1 | GAGAACAGCAAGTGCACCAA | 43 | TTGGAATCTGGAGATGGAGG | 44 |
| Liprinβ1 isoform2 | AAAGGCTGGCACGTTTAGAA | 45 | AGGGAAATCCCATCTTGGTT | 46 |
| SULF2 | CATCGACCACGAGATTGAAA | 41 | CCGCTTTTTCTTCAGGTGAC | 42 |
| EIF1AX Isoforms 1 + 2 | CCGGAAAGAAGTCAGAGACG | 47 | TTGCTTCTAGCCGTCCATTT | 48 |
| EIF1AX isoform 1 | GAAAGAAGTCAGAGACGCCG | 49 | TTGCTTCTAGCCGTCCATTT | 50 |
| PLTP Isoforms 1 + 2 | CATGAAGGATCCTGTGGCTT | 51 | CAGGACAATGCTCCCAAAGT | 52 |
| PLTP isoform 1 | AGTGTCCAATGTCTCCTGCC | 53 | CAACAAGCTCGTCCACAGAA | 54 |

Transfection of Small Interference RNA (siRNA)

Interference RNA (siRNA) are small RNA, which recognise by complementarity a sequence on the targeted mRNA and enable their degradation. Eurogentec proposed the siRNA design which we then tested to determine their efficacy, the effective siRNA concentrations and the necessary culture time (table 2).

TABLE 2
List of the siRNA duplexes designed (the complementary
sequences are not described). The validated and selected
siRNA are indicated in bold.

| Extinction | | | | |
|---|---|---|---|---|
| predie | Nom | Position | siRNA Sequence (5′ -> 3′) | Lenght |
| 2 siRNA = | ORP4#1 | 2046 | CCUCAACUGUUCACAACAU* | 19 |
| 70% | | | | |
| | ORP4#2 | 1155 | GAGAUACACAGUCGGAAAU* | 19 |
| 2 siRNA = | SULF2#1 | 2655 | CUGGCUUCCUAGAGUACUU* | 19 |
| 70% | | | | |
| | SULF2#2 | 1575 | GAGGCAAGCUGCUACACAA* | 19 |
| 2 siRNA = | GALNT1#1 | 463 | GACACAUGAUAGAAGAAAU* | 19 |
| 70% | | | | |
| | GALNT1#2 | 928 | GAGAUUACUUUCAGGAAAU* | 19 |

Extinction prédie: Predicted Extinction; Nom: Name

The transfection is carried out by means of Effecten® (Quioagen, Courtabeuf, France), which showed the best transfection efficacy compared to Lipofectamine® (Invitrogen) and the transfection kit marketed by Cell Signalling. The day before the transfection, the cells are trypsinised and placed in culture at 75%-80% of confluence (i.e. 75,000 cells on a 12 well assay plate). The cells are transfected with the siRNA at a concentration of 100 nM, in a volume of 0.5 ml for 6 hours, the medium is then removed and replaced by 1 ml of 10% SVF medium. The cells are cultivated for 54 hours before carrying out the functional apoptosis tests.

### Measurement of Phospholipid Transfer Activity (FIG. 7)

We detected an increased activity of PLTP in synovial fluids of RA patients in comparison with those of contoral patients (OA, ie osteoarthritis). This strongly suggests a role of PLTP in RA. Phospholipid transfer activity was measured using a commercially available fluorescence activity assay (Cardiovascular targets, New York, N.Y., USA) following the instructions provided by the manufacturer. The PLTP Activity Kit includes donor and acceptor particles. Incubation of donor and acceptor with PLTP source results in the PLTP-mediated transfer of fluorescent phospholipid. The fluorescent phospholipid (NBD-labelled phospholipid) is present in a self-quenched state when associated with the donor. PLTP-mediated transfer is determined by the increase in fluorescence intensity as the fluorescent lipid is removed from the donor and transferred to the acceptor. Briefly, serum samples (5 μl), fluorescent-labelled donors (3 μl) and unlabelled acceptors (50 μl), were incubated at 37° C. in a final volume of 100 μl of TBS in 96 well microplates. Changes in fluorescence were monitored every minute using a Victor2™ fluorescent counter (PerkinElmer Life Sciences) for a 30 min period, with a 465 nm excitation and a 535 nm emission wavelength. PLTP activity in seminal plasma (increase in fluorescence) was calculated as the increase in fluorescence between 0 and 20 min. Initial phospholipid transfer rates (increase in fluorescence/min) were calculated by dividing the increase in fluorescence in the samples between 0 and 5 min by the incubation time.

### Results

### 1. Patients

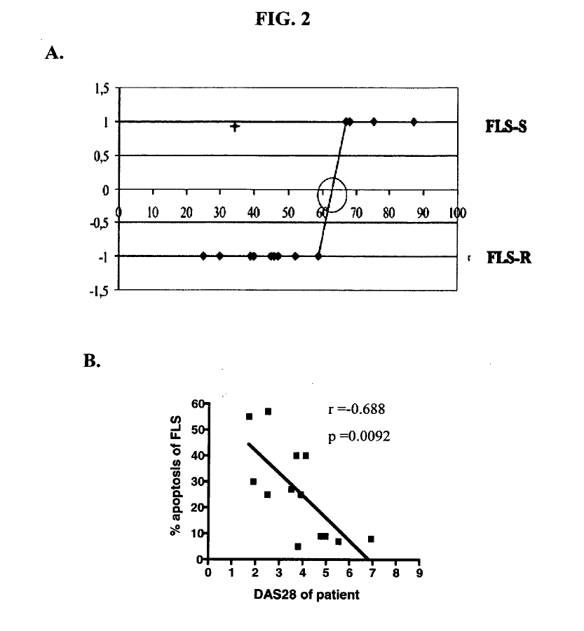

When investigating the age and sex of the patients, it appears that the FLS which are resistant to TRAIL-induced apoptosis are mainly isolated from synovial tissue biopsies of women aged under 60 years (table 3, FIG. 2A). Only one FLS-S culture is isolated from the tissue of a woman aged less than 60 years (F, 36 years, see table 3, in bold). Five other FLS-S are isolated from biopsies of women aged 60 years or over, and the 4 remaining ones from biopsies of men aged under 60 years. Six of the 7 FLS cultures obtained from biopsies of men have high (4/7) or intermediate (2/7) sensitivity.

TABLE 3
Sex and age of the patients at the time of collecting the synovial
tissue biopsy from which the FLS will be isolated.

| Sample | Sex | Age |
|---|---|---|
| RAFLS-R1 | F | 52 |
| RAFLS-R2 | F | 46 |
| RAFLS-R3 | F | 30 |
| RAFLS-R4 | F | 59 |
| RAFLS-R5 | F | 45 |
| RAFLS-R6 | F | 39 |
| RAFLS-R7 | F | 47 |
| RAFLS-R8 | H | 57 |
| RAFLS-R9 | F | 40 |
| RAFLS-R10 | F | 25 |
| RAFLS-R11 | F | 46 |
| RAFLS-S1 | F | 68 |
| RAFLS-S2 | H | 50 |
| RAFLS-S3 | F | 36 |
| RAFLS-S4 | F | 67 |
| RAFLS-S5 | H | 55 |
| RAFLS-S6 | F | 87 |
| RAFLS-S7 | F | 60 |
| RAFLS-S8 | H | 55 |
| RAFLS-S9 | H | 57 |
| RAFLS-S10 | F | 64 |
| RAFLS-I1 | H | 63 |
| RAFLS-I2 | F | 57 |
| RAFLS-I3 | F | 75 |
| RAFLS-I4 | H | 76 |
| RAFLS-I5 | F | 63 |

In addition, we observed that TRAIL-sensitivity of RA FLS varies according to the patients they derive from. Synovial fibroblasts from some patients are nearly resistant to apoptosis when exposed to TRAIL, but respond with increased proliferation in comparison with untreated cells (FIG. 2B). Noteworthy, FLS resistant to TRAIL-induced apoptosis derived from patients with more severe disease symptoms than those of TRAIL-sensitive FLS. Moreover, sensitivity of FLS towards TRAIL-induced apoptosis inversely correlated with the index of disease activity of rheumatoid arthritis patients (DAS28). Thus, TRAIL-responses of synovial fibroblast appear to correlate with disease severity.

### 2. Candidate Genes Determined by the Microarray Technique

The collection of FLS is dependent on the frequency of synovial tissue biopsies obtained. We therefore chose to perform a first experiment relating to 6 FLS per group, even though we initially planned to use at least 10 FLS in each group. Their sensitivity is set out in table 4.

TABLE 4
Sensitivity of the FLS used for the transcriptome analysis by
microarray

| | |
|---|---|
| FLS-R | FLS-S |

| | | | |
|---|---|---|---|
| RAFLS-R1 | 7% | FLS-S1 | 40% |
| RAFLS-R2 | 10% | FLS-S2 | 30% |
| RAFLS-R3 | 5% | FLS-S3 | 25% |
| RAFLS-R4 | 5% | FLS-S4 | 45% |
| RAFLS-R5 | 8% | FLS-S5 | 30% |
| RAFLS-R6 | 9% | FLS-S6 | 50% |

The DNA chip technique makes it possible to control the expression level of a large number of genes. A differential analysis revealed 12 factors differentially expressed between cells resistant to TRAIL-induced apoptosis and sensitive cells (table 5). The oligos detected with the microarray are listed in the sequence listing as SEQ ID NO:23 to SEQ ID NO:35 (see also table 6). The candidates are classed according to the probability of their being significantly differentially expressed between the two groups of FLS. These factors are implicated in various functions, in particular in the respiratory chain (ATPase 6, NADH 3), in the transportation or metabolism of lipids (ORP-4, Phosopholipid transfer protein II) and in the regulation of signalling linked to extracellular factors (Sulfatase 2, GalNac-T1, Sialate OAE, Liprin β1). The functions of PRAME family of genes (for instance PRAME 5, 3, 9, 18 and 19), Acheron, eIF-1A and TET-1 are not well known. Sialate OAE and especially PRAME have the benefit of being associated with tumours, however, tumour cells are the privileged targets of TRAIL.

Three of the candidate genes identified during the comparison of the transcriptome of the FLS-R and FLS-S intervene in the glycosylation mechanisms: GALNT-1, SULF-2 and SIAL. Glycosylation is a modification of proteins and lipids which helps to substantially modulate the cellular mechanisms, such as adhesion, receptor activation, intracellular signalling. In addition, glycosylated proteins are often associated with lipid rafts, which are important platforms for the regulation of the signalling of numerous receptors, in particular of TRAIL receptors. However, ORP-4 is a protein which controls the metabolism of lipids, in particular of cholesterol and ceramides, which themselves form part of the composition of lipid rafts.

TABLE 5
Genes deriving from the comparison of the transcriptome by microarray.

| gene ID | official symbol | Official full name | Function | P. | Chromosome |
|---|---|---|---|---|---|
| 57667 | KIAA1546 | tet oncogene family | Unknown | 99.8 | 4q24 |
| (new ID: | | member 2 (TET2) | — | | |
| 54790) | | | | | |
| 23762 | OSBP2/ORP-4 | ORP-4 Oxysterol- | Oxysterols are byproducts of | 99.4 | 22q12.2 |
| | | binding protein 2 | cholesterol that can have | | |
| | | (Oxysterol binding | cytotoxic effects on many cell | | |
| | | protein-related | types. The membrane-bound | | |
| | | protein 4) | protein encoded by this gene | | |
| | | | contains a pleckstrin | | |
| | | | homology (PH) domain and | | |
| | | | an oxysterol-binding region. It | | |
| | | | binds oxysterols such as 7- | | |
| | | | ketocholesterol and may | | |
| | | | inhibit their cytotoxicity. | | |
| 54414 | SIAE | sialic acid | Sialic acids are acidic 9-carbon | 91.5 | 11q24 |
| | | acetylesterase | sugars typically found at the | | |
| | | (cytosolic sialic acid | nonreducing end of sugar | | |
| | | 9-O-acetylesterase | chains. They are frequently | | |
| | | homolog) | modified by 9-O-acetylation, | | |
| | | | and this modification is | | |
| | | | removed by sialic acid | | |
| | | | acetylesterases. | | |
| 8496 | LIPRINβ1 | PTPRF interacting | The protein encoded by this | 91.0 | 12p11.23-p11.22 |
| | | protein, binding | gene is a member of the LAR | | |
| | | protein 1 (liprin | protein-tyrosine phosphatase- | | |
| | | beta 1) | interacting protein (liprin) | | |
| | | | family. Liprins interact with | | |
| | | | members of LAR family of | | |
| | | | transmembrane protein | | |
| | | | tyrosine phosphatases, which | | |
| | | | are known to be important for | | |
| | | | axon guidance and mammary | | |
| | | | gland development. It has | | |
| | | | been proposed that liprins are | | |
| | | | multivalent proteins that form | | |
| | | | complex structures and act as | | |
| | | | scaffolds for the recruitment | | |
| | | | and anchoring of LAR family | | |
| | | | of tyrosine phosphatases. This | | |
| | | | protein was found to interact | | |
| | | | with S100A4, a calcium- | | |
| | | | binding protein related to | | |
| | | | tumor invasiveness and | | |
| | | | metastasis. In vitro | | |
| | | | experiment demonstrated that | | |
| | | | the interaction inhibited the | | |
| | | | phosphorylation of this | | |
| | | | protein by protein kinase C | | |
| | | | and protein kinase CK2. | | |
| | | | Alternatively spliced | | |
| | | | transcript variants encoding | | |
| | | | distinct isoforms have been | | |
| | | | reported (e.g. isoforms 1&2). | | |
| 4508 | MT-ATP6 | mitochondrially | ATP synthesis | 88.9 | mitochondrion |
| | | encoded ATP | | | |
| | | synthase 6 | | | |
| 55959 | SULF2 | Sulfatase 2 | Heparan sulfate proteoglycans | 86.1 | 20q12-q13.2 |
| | | | (HSPGs) act as coreceptors for | | |
| | | | numerous heparin-binding | | |
| | | | growth factors and cytokines | | |
| | | | and are involved in cell | | |
| | | | signaling. Heparan sulfate 6-O- | | |
| | | | endosulfatases, such as SULF2, | | |
| | | | selectively remove 6-O-sulfate | | |
| | | | groups from heparan sulfate. | | |
| | | | This activity modulates the | | |
| | | | effects of heparan sulfate by | | |
| | | | altering binding sites for | | |
| | | | signaling molecules | | |
| 1964 | EIF1AX | eukaryotic | essential eukaryotic | 74.4 | Xp22.12 |
| | | translation | translation initiation factor. | | |
| | | initiation factor 1A, | Alternatively spliced | | |
| | | X-linked | transcript variants encoding | | |
| | | | distinct isoforms have been | | |
| | | | reported (e.g. isoforms 1&2). | | |
| | PRAMEF5 | PRAME family | Unknown | 72.8 | 1p36.21 |
| | | member (oligo | | | |
| | | matches several | | | |
| | | family members, | | | |
| | | including 3, 9, 18, | | | |
| | | 19) | | | |
| 4537 | MT-ND3 | mitochondrially | enzyme located in the inner | 71.3 | mitochondrion |
| | | encoded NADH | mitochondrial membrane that | | |
| | | dehydrogenase 3 | catalyzes the transfer of | | |
| | | | electrons from NADH to | | |
| | | | coenzyme Q | | |
| 55323 | LARP6 | La | unknown, possibly involved in | 68.4 | 15q23 |
| | (Acheron) | ribonucleoprotein | cell death. Alternatively | | |
| | | domain family, | spliced transcript variants | | |
| | | member 6 | encoding distinct isoforms | | |
| | | | have been reported (e.g. | | |
| | | | isoforms 1&2). | | |
| 5360 | PLTP | phospholipid transfer | The encoded protein transfers | 65.1 | 20q12-q13.1 |
| | | protein | phospholipids from triglyceride- | | |
| | | | rich lipoproteins to high density | | |
| | | | lipoprotein (HDL). In addition | | |
| | | | to regulating the size of HDL | | |
| | | | particles, this protein may be | | |
| | | | involved in cholesterol | | |
| | | | metabolism. Alternatively | | |
| | | | spliced transcript variants | | |
| | | | encoding distinct isoforms have | | |
| | | | been reported (e.g. isoforms | | |
| | | | 1&2). | | |
| 2589 | GALNT1 | UDP-N-acetyl-alpha- | GalNAc-Ts initiate mucin-type | 64.5 | 18q12.1 |
| | | D- | O-linked glycosylation in the | | |
| | | galactosamine:polypeptide | Golgi apparatus by catalyzing | | |
| | | N- | the transfer of GalNAc to serine | | |
| | | acetylgalactosaminyl | and threonine residues on target | | |
| | | transferase 1 | proteins. GALNT14, an isoform | | |
| | | (GalNAc-T1) | of the GALTN1 has been | | |
| | | | recently shown to modulate | | |
| | | | TRAIL-responsiveness in tumor | | |
| | | | cell lines (Wagner et al, 2007, | | |
| | | | Nat Med 13: 1070-1077) | | |

Result of the gene expression analysis of synovial fibroblasts of rheumatoid arthritis (RA FLS) patients being either resistant or susceptible towards TRAIL induced apoptosis.
The table shows those genes that are differentially expressed between the two groups of fibroblasts with a probability of at least 64%.
Genes in bold are overexpressed in TRAIL resistant RA FLS, those not in bold are overexpressed in TRAIL sensitive RA FLS. (P: probability)

### 3. Validation of the Candidates

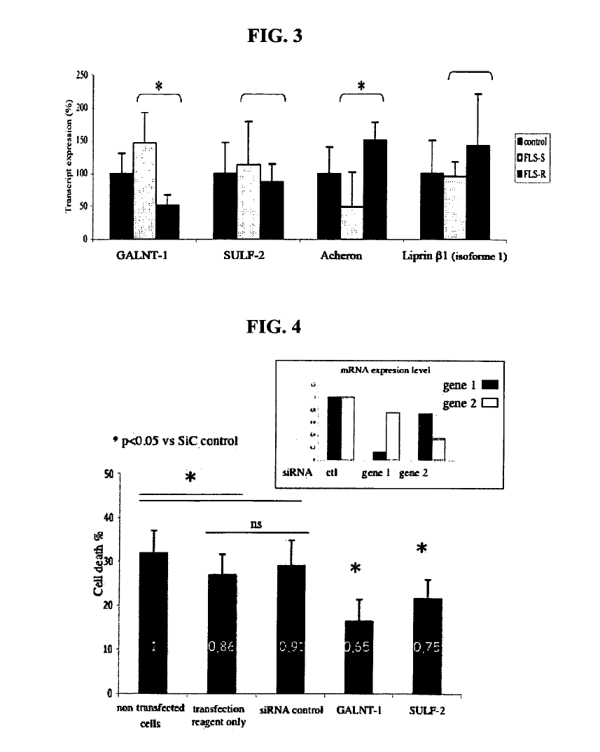

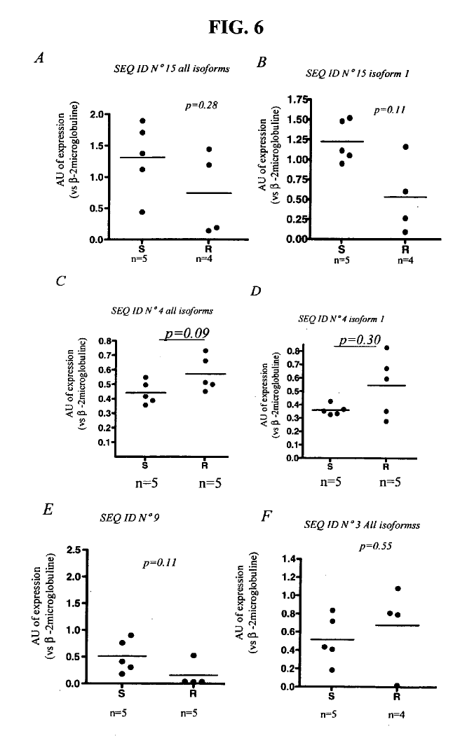

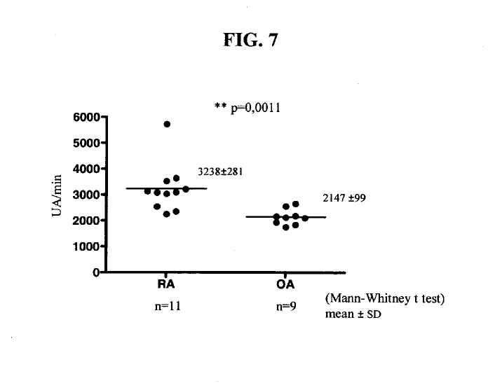

Among the 12 genes or family of genes (PRAME) deriving from the statistical analysis, we first selected 4 candidate genes, Sulfatase 2 (SULF-2), GalNT Transferase 1 (GALNT-1), Liprin J31, and Acheron (LARP6), which seemed to us to be of interest in the question of cell survival and cell death in response to TRAIL. We proceeded to verify the differential expression by quantitative RT-PCR (RT-QPCR). Our experiments show that GALNT-1 and SULF-2 and PLTPtend to be overexpressed in the FLS-S; Acheron and Liprin β1 and EiF1A in the FLS-R (FIGS. 3 and 6). Moreover, the increased activity of PLTP found in synovial fluids of RA patients underlines its importance in this disease (FIG. 7).

### 4. Effect of the Extinction of Candidate Genes on TRAIL-Induced Apoptosis

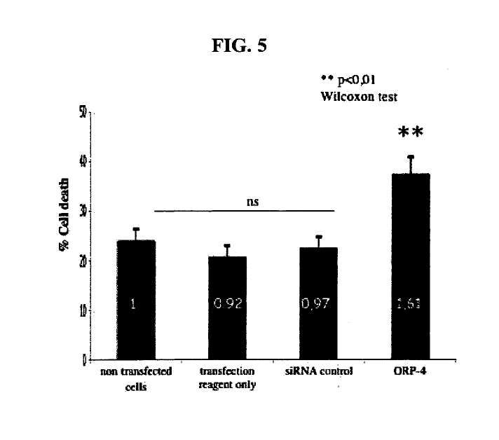

The functionality and influence of the candidates on TRAIL-induced apoptosis is verified by using the siRNA method, thereby making it possible to extinguish the expression of proteins corresponding to candidate genes or by transfection of vectors which enable their overexpression. We designed siRNAs for 3 candidates (ORP-4, GALNT-1 and SULF-2) and evaluated their effect on extinction these genes to verify their role in the control of TRAIL-induced apoptosis. Preliminary experiments enabled us to define the best conditions for transfection. The efficacy of the extinction of the expression of GALNT-1 and SULF-2 genes is verified by quantitative PCR. With regard to SULF-2 and GALNT-1, we were able to verify the extinction of their expression at mRNA level and this reduction is around 80-90% (box, FIG. 4). Concerning ORP-4, we were able to extinguish its expression by around 50% (box, FIG. 5). The siRNAs which target the GALNT-1 and SULF-2 genes significantly diminished the TRAIL-induced apoptosis of the FLS-S, to 67% and 75% respectively compared to TRAIL-induced apoptosis in the non transfected FLS (FIG. 4). Neither the control siRNA nor the transfection reagent significantly modify TRAIL-induced apoptosis (FIG. 4). Neither the control siRNA nor the transfection reagent significantly modify TRAIL-induced apoptosis (FIGS. 4 and 5). On the other hand, the reduction in ORP-4 significantly increases TRAIL-induced apoptosis to 167% compared to non transfected cells (FIG. 5).

### 5. Conclusion

In order to determine the molecular factors which differentiate the FLS-S from the FLS-R, we undertook a comparison of the transcriptome of the two groups by the DNA chip technique, which enables us to compare the expression of a wide panel of genes. The latter enabled us to identify 12 differentially expressed genes or family of genes (PRAME). Among these, we have tested the functionality of 3 genes, GALNT-1, SULF-2 and ORP-4 by the siRNA technique. The reduction in the expression of the targeted genes seems to be sufficient to observe a cellular effect since the siRNA which target GALNT-1, SULF-2 and ORP-4 significantly influence TRAIL-induced apoptosis, with a cell death of 67%, 75% and 167% respectively, compared to the TRAIL-induced apoptosis of non transfected cells. GALNT-1 and SULF-2 are thus factors which participate in TRAIL-induced apoptosis whereas ORP-4 participate to the resistance against TRAIL-induced apoptosis.

### SEQUENCE LISTING

TABLE 6
Identification of the nucleotide sequences of the
invention by their SEQ IDs in the sequence listing.

| | Gene nucleotide | Oligo used in | Primer used | |
|---|---|---|---|---|
| Name | sequence | the microarray | for QPCR | Si RNA |
| TET2 | SEQ ID NO: 1 | SEQ ID NO: 23 | | |
| ORP-4 | SEQ ID NO: 2 | SEQ ID NO: 24 | | SEQ ID NO: 17 and |
| | | | | SEQ ID NO: 18 |
| LIPRINβ1 | SEQ ID NO: 3 | SEQ ID NO: 26 | SEQ ID NO: 39 and | |
| | | | SEQ ID NO: 40 for | |
| | | | isoform 1; | |
| | | | SEQ ID NO: 43 and | |
| | | | SEQ ID NO: 44 for | |
| | | | isoform 1; | |
| | | | SEQ ID NO: 45 and | |
| | | | SEQ ID NO: 46 for | |
| | | | isoform 2 | |
| EIF1AX | SEQ ID NO: 4 | SEQ ID NO: 29 | SEQ ID NO: 47 and | |
| | | | SEQ ID NO: 48 for | |
| | | | isoforms 1 and 2; | |
| | | | SEQ ID NO: 49 and | |
| | | | SEQ ID NO: 50 for | |
| | | | isoform 1 | |
| LARP6 | SEQ ID NO: 5 | SEQ ID NO: 31 | SEQ ID NO: 35 and | |
| | | | SEQ ID NO: 36 | |
| SIAE | SEQ ID NO: 6 | SEQ ID NO: 25 | | |
| MT-ATP6 | SEQ ID NO: 7 | SEQ ID NO: 27 | | |
| MT-ND3 | SEQ ID NO: 8 | SEQ ID NO: 32 | | |
| SULF2 | SEQ ID NO: 9 | SEQ ID NO: 28 | SEQ ID NO: 41 and | SEQ ID NO: 19 and |
| | | | SEQ ID NO: 42 | SEQ ID NO: 20 |
| PRAME5 | SEQ ID NO: 10 | SEQ ID NO: 30 | | |
| PRAME3 | SEQ ID NO: 11 | SEQ ID NO: 30 | | |
| PRAME9 | SEQ ID NO: 12 | SEQ ID NO: 30 | | |
| PRAME18 | SEQ ID NO: 13 | SEQ ID NO: 30 | | |
| PRAME19 | SEQ ID NO: 14 | SEQ ID NO: 30 | | |
| PLTP | SEQ ID NO: 15 | SEQ ID NO: 33 | SEQ ID NO: 51 and | |
| | | | SEQ ID NO: 52 for | |
| | | | isoforms 1 and 2; | |
| | | | SEQ ID NO: 53 and | |
| | | | SEQ ID NO: 54 for | |
| | | | isoform 1 | |
| GALNT1 | SEQ ID NO: 16 | SEQ ID NO: 34 | SEQ ID NO: 37 and | SEQ ID NO: 21 and |
| | | | SEQ ID NO: 38 | SEQ ID NO: 22 |

### REFERENCES

Throughout this application, various references describe the state of the art to which this invention pertains. The disclosures of these references are hereby incorporated by reference into the present disclosure.

1. A method for identifying genes involved in TRAIL-induced apoptosis in a population of cells comprising the steps of:
1) contacting said population of cells with TRAIL,
2) isolating the subset of cells of the population which are sensitive to TRAIL-induced apoptosis (sensitive subset) and the subset of cells of the population which are resistant to TRAIL-induced apoptosis (resistant subset),
3) comparing the gene expression in the sensitive subset and in the resistant subset, and
4) identifying the genes that are differentially expressed in the sensitive subset and in the resistant subset, the genes being over expressed in the sensitive subset being classified as genes sensitizing the cells of said population to TRAIL-induced apoptosis and the genes being over expressed in the resistant subset being classified as genes inducing resistance of the cells of said population to TRAIL-induced apoptosis.

2. An inhibitor of the expression of a gene inducing resistance of cells to TRAIL-induced apoptosis, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5.

3. The inhibitor according to claim 2, wherein said inhibitor is a siRNA comprising a nucleotide sequence as shown in SEQ ID NO:17 or SEQ ID NO:18.

4. An activator of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

5. An isolated nucleotide sequence selected from the group comprising SEQ ID NO:17, SEQ ID NO:18, SEQ ID NO:19, SEQ ID NO:20, SEQ ID NO:21 and SEQ ID NO:22.

6. A method for sensitizing cells to TRAIL-induced apoptosis, said method comprising the step of contacting said cells with a product capable of sensitizing cells to TRAIL-induced apoptosis, wherein said product is selected from the group comprising:
inhibitors of the expression of a gene inducing resistance of cells to TRAIL, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5
activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16;
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

7. (canceled)

8. A method for treating a hyperproliferative disease in a human or animal body, comprising administering to said human or animal an effective amount of a product capable of sensitizing cells to TRAIL-induced apoptosis wherein said product is selected from the group comprising:
inhibitors of the expression of a gene inducing resistance of cells to TRAIL, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5
activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

9. The method according to claim 8, wherein the hyperproliferative disease is selected from the group comprising cancer and rheumatoid arthritis.

10. The method according to claim 8, wherein said method further comprises the simultaneous, sequential or separate administration of an effective amount of TRAIL in said human or animal body.

11. A method for determining the responsiveness of a subject suffering from a hyperproliferative disease to TRAIL, comprising the step of detecting, in hyperproliferative cells obtained from said subject:
a) the expression of a gene inducing resistance of said cells to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, and wherein the detection of the expression of a gene inducing resistance of said cells to TRAIL-induced apoptosis indicates that said subject is not responsive to TRAIL,
and/or
b) the expression of a gene sensitizing said cells to TRAIL-induced apoptosis wherein said gene comprises a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and wherein the detection of the expression of a gene sensitizing said cells to TRAIL-induced apoptosis indicates that said subject is responsive to TRAIL.

12. (canceled)

13. A pharmaceutical composition comprising a product capable of sensitizing cells to TRAIL-induced apoptosis, together with a pharmaceutically acceptable carrier, wherein said product is selected from the group comprising:
inhibitors of the expression of a gene inducing resistance of cells to TRAIL, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5
activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

14. The pharmaceutical composition according to claim 13, wherein said pharmaceutical composition further comprises TRAIL.

15. The method according to claim 1, wherein said cells are hyperproliferative cells selected from the group comprising cancer cells and rheumatoid arthritis fibroblast-like synoviocytes.

16. A method for determining the prognosis of a subject suffering from a hyperproliferative disease, comprising the step of detecting, in a sample obtained from said subject:
a) the expression of a gene inducing resistance to TRAIL-induced apoptosis wherein said gene comprises a nucleotide sequence as shown in SEQ ID NO:1, SEQ ID NO:2, SEQ ID NO:3, SEQ ID NO:4 or SEQ ID NO:5 or comprises a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5, wherein said expression indicates that the subject has a poor prognosis,
and/or
b) the expression of a gene sensitizing said cells to TRAIL-induced apoptosis wherein said gene comprises a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprises a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, wherein said expression indicates that the subject has a good prognosis.

17. (canceled)

18. A method for sensitizing cells to TRAIL-induced apoptosis in a human or animal body, said method comprising administering an effective amount of a product selected from the group comprising:
inhibitors of the expression of a gene inducing resistance of cells to TRAIL, said gene comprising a nucleotide sequence as shown in SEQ ID NO:1, SEQ ID NO:2, SEQ ID NO:3, SEQ ID NO:4 or SEQ ID NO:5 or comprising a nucleotide sequence having at least 70% of identity, particularly at least 80% of identity, more particularly at least 90% identity with a nucleotide sequence selected from the group consisting of SEQ ID NO:1, SEQ ID NO:2, SEQ ID NO:3, SEQ ID NO:4 and SEQ ID NO:5
activators of the expression of a gene sensitizing cells to TRAIL-induced apoptosis, said gene comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16,
expression vectors comprising a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or comprising a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, and
proteins able to sensitize cells to TRAIL-induced apoptosis, said proteins being encoded by a nucleotide sequence as shown in SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16 or by a nucleotide sequence having at least 70% of identity, particularly at least 80% of identity, more particularly at least 90% identity with a nucleotide sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

19-20. (canceled)

21. The method according to claim 18, wherein said cells are hyperproliferative cells selected from the group comprising cancer cells and rheumatoid arthritis fibroblast-like synoviocytes.

22. The product according to claim 8, wherein said cells are hyperproliferative cells selected from the group comprising cancer cells and rheumatoid arthritis fibroblast-like synoviocytes.

23-25. (canceled)

26. The pharmaceutical composition according to claim 13, wherein said cells are hyperproliferative cells selected from the group comprising cancer cells and rheumatoid arthritis fibroblast-like synoviocytes.

27. (canceled)

28. The method according to claim 11, wherein said cells are hyperproliferative cells selected from the group comprising cancer cells and rheumatoid arthritis fibroblast-like synoviocytes.
